(19) United States
(12) Patent Application Publication
(10) Pub. No.: US 2024/0182568 A1
(43) Pub. Date: Jun. 6, 2024

(54) METHODS OF ENHANCING ANTIBODY THERAPIES

(71) Applicant: UNIVERSITY OF IOWA RESEARCH FOUNDATION, Iowa City, IA (US)

(72) Inventors: George WEINER, Iowa City, IA (US); Zhaoming WANG, Iowa City, IA (US)

(73) Assignee: UNIVERSITY OF IOWA RESEARCH FOUNDATION, Iowa City, IA (US)

(21) Appl. No.: 18/279,380

(22) PCT Filed: Mar. 8, 2022
(86) PCT No.: PCT/US2022/019357

§ 371 Date: Aug. 29, 2023

Related U.S. Application Data
(60) Provisional application No. 63/159,276, filed on Mar. 10, 2021.

(51) Int. Cl. C07K 16/28; A61K 39/00; A61K 45/06; A61P 35/00; C07K 16/30

(52) U.S. Cl. CPC C07K 16/2809; A61K 45/06; A61P 35/00; C07K 16/2803; C07K 16/30; A61K 2039/507; A61K 2039/545; C07K 2317/31; C07K 2317/732

(57) ABSTRACT

The invention provides methods for treating a hyperproliferative disorder in a mammal in need thereof comprising, administering to the mammal a combination of (a) an anti-cancer antibody and (b) a T cell activating agent for the prophylactic or therapeutic treatment of the hyperproliferative disorder. The invention further provides methods for maintaining long-term natural killer (NK) cell antibody-dependent cellular cytotoxicity (ADCC) in the treatment of a hyperproliferative disorder in a mammal in need thereof comprising, administering to the mammal a combination of (a) an anti-cancer antibody, and (b) a T cell activating agent for the prophylactic or therapeutic treatment of the hyperproliferative disorder.

### CROSS-REFERENCE TO RELATED APPLICATION

This application claims priority to U.S. Provisional Application Ser. No. 63/159,276, filed on Mar. 10, 2021, the disclosure of which is hereby incorporated herein by reference in its entirety.

### STATEMENT REGARDING FEDERALLY SPONSORED RESEARCH

This invention was made with government support under CA097274 awarded by National Institutes of Health. The government has certain rights in the invention.

### BACKGROUND

Therapeutic anti-tumor monoclonal antibody (mAb) treatments have become a standard and important component of cancer therapy starting with FDA approval of rituximab (RTX), a mouse-human chimeric anti-CD20 mAb, in 1997. Some B cell malignancies, and other cancers that respond to mAb therapy, fail to respond or recur following anti-CD20-based or other antibody-based therapy. Accordingly, new treatments are needed for treatment-resistant hyperproliferative disorders.

### SUMMARY

It has been determined that additive or synergistic effects in inhibiting the growth of cancer cells in vitro can be achieved by administering an anti-cancer antibody in combination with an T cell activating agent. The combinations and methods may be useful in the treatment of hyperproliferative disorders such as cancer.

One aspect of the invention provides a method for treating a hyperproliferative disorder in a mammal comprising, administering to the mammal, (a) anti-cancer antibody; and (b) one or more agents selected from a bispecific antibody, Chimeric antigen receptor T (CAR-T) cells, and/or vaccines that induce a T cell response.

One aspect of the invention provides a method for maintaining long-term natural killer (NK) cell antibody-dependent cellular cytotoxicity (ADCC) in the treatment of a hyperproliferative disorder in a mammal in need thereof comprising, administering to the mammal a combination of (a) an anti-cancer antibody, and (b) a T cell activating agent for the prophylactic or therapeutic treatment of the hyperproliferative disorder.

### BRIEF DESCRIPTION OF DRAWINGS

FIGS. 1A-1G. Long-term exposure to RTX impacts NK cell function and phenotype. PBMC were cocultured with Raji cells and RTX or TRA. NK cell function and phenotype were examined at different time points. (FIG. 1A) CD19⁺ target cells are progressively eliminated over time. (FIG. 1B) RTX maintains the number of NK cells within PBMCs. (FIG. 1C) RTX induces proliferation of NK cells within PBMC at 7 days as determined by CFSE dilution. (FIGS. 1D, 1E) RTX progressively increases the percent of CD56^{bright} NK cells beginning at day. n=6. Cell counts at 0 hour were used to normalize cell numbers. (FIG. 1F) Patient 4 has circulating tumor cells. The other patients have no detectable circulating tumor cells. (FIG. 1G) The fraction of CD56^{bright} NK cells increased following RTX in the patient with circulating malignant cells but not in patients without circulating malignant cells.

FIGS. 2A-2I. CD56^{dim} NK cells transit into CD56^{bright} NK cells in response to long-term RTX activation. (FIG. 2A) Isolated CD56^{dim} and CD56^{bright} NK cells were stained with CFSE and CellTracker Red respectively. CFSE-labeled CD56^{dim} and Red-labeled CD56^{bright} NK were added to autologous PBMC and cocultured with Raji cells and RTX or TRA. (FIG. 2B) As illustrated by a day 7 histogram gated on CFSE-labeled CD56^{dim} NK cells, CFSE-labeled CD56^{dim} NK cells proliferate and adopt a CD56^{bright} phenotype in the RTX group. n=3. (FIG. 2C) The number of CellTracker Red labeled-CD56bright NK cells does not increase in response to RTX. n=3. (FIGS. 2D-2I) CD16, CD57 and KIR were largely expressed by resting CD56dim NK cells. RTX-induced CD56^{bright} NK cells express high levels of CD16, CD57 and KIR. n=6. Cell counts at 0 hour were used to normalize cell numbers.

FIGS. 3A-3G. T cells are required for RTX-mediated NK cell cytotoxicity, viability, CD16 re-expression and CD56^{dim} to CD56^{bright} transition. Unfractionated PBMC or PBMC depleted of CD3⁺, CD4⁺, or CD8⁺ cells were cocultured with Raji cells and RTX or TRA. (FIGS. 3A, 3B) CD19⁺ target cells are mostly eliminated by RTX on day 7. RTX-mediated elimination of CD19⁺ cells is inhibited by the depletion of CD3⁺ T cells. (FIG. 3C) The number of NK cells remaining in intact PBMCs is maintained by RTX but not by TRA. However, NK cell numbers are not maintained after CD3⁺ T cell depletion. (FIGS. 3D, 3E) The expression of CD16 is downregulated on NK cells by RTX activation at 20 h and recovers on day 7. CD16 re-expression is not observed after CD3⁺ T cell depletion. (FIGS. 3F, 3G) CD56^{dim} to CD56^{bright} NK transition is induced in unfractionated PBMC after 7 days. This transition is inhibited by depletion of CD3⁺ or CD4⁺ cells but not after depletion of CD8⁺ cells. Cell counts in the TRA+PBMC group were used to normalize cell numbers. n=4-6. Dep: depleted.

FIGS. 4A-4G. T cells are required for RTX-mediated NK cell responses in the autologous system. Unfractionated PBMC or PBMC depleted of CD3⁺ cells were cocultured with enriched numbers of autologous B cells and TRA or RTX. RTX-mediated elimination of CD19⁺ autologous B cells (FIGS. 4A, 4B), the number of NK cells (FIG. 4C), CD56^{dim} to CD56bright transition (FIGS. 4D, 4E) and CD16 re-expression (FIGS. 4F, 4G) on NK cells is suppressed by the depletion of CD3⁺ T cells after 7 days. Cell counts in the TRA+PBMC group were used to normalize cell numbers. n=7-8. Dep: depleted.

FIGS. 5A-5L. T cells impact RTX-mediated NK cell responses in a contact dependent manner. CD3⁺ T cells were depleted from PBMC and then added back to the lower Transwell chamber (with Raji and remaining PBMCs) or the upper chamber (separated from Raji and remaining PBMCs), then cultured with RTX or TRA for 7 days. (FIGS. 5A-5D) Elimination of CD19⁺ target cells, the number of NK cells, CD56^{dim} to CD₅₆^{bright} NK transition and CD16 recovery by RTX activation is suppressed by the physical separation of T cells (CD3^{Trans}) from the remainder of the PBMCs. n=5.

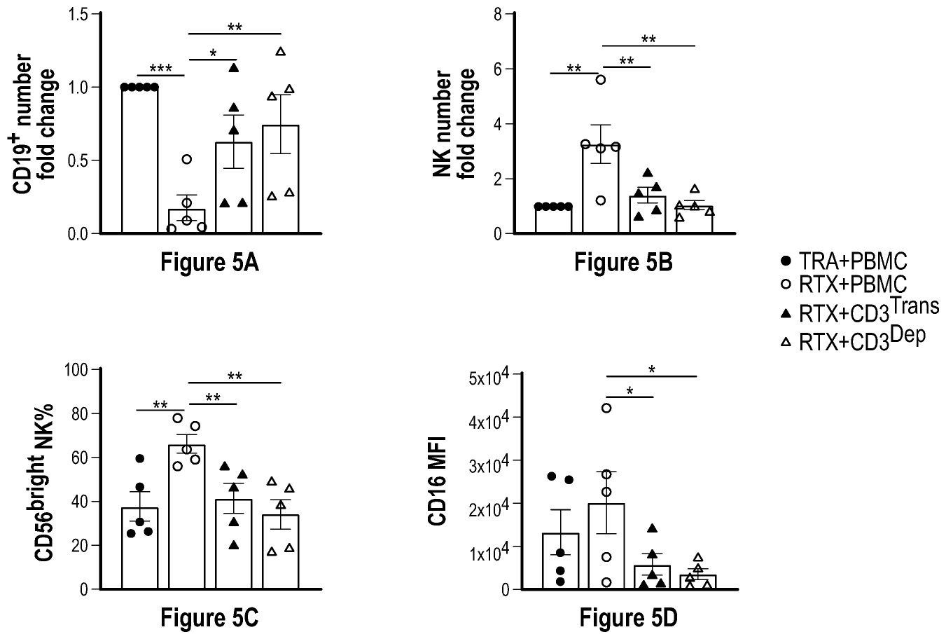

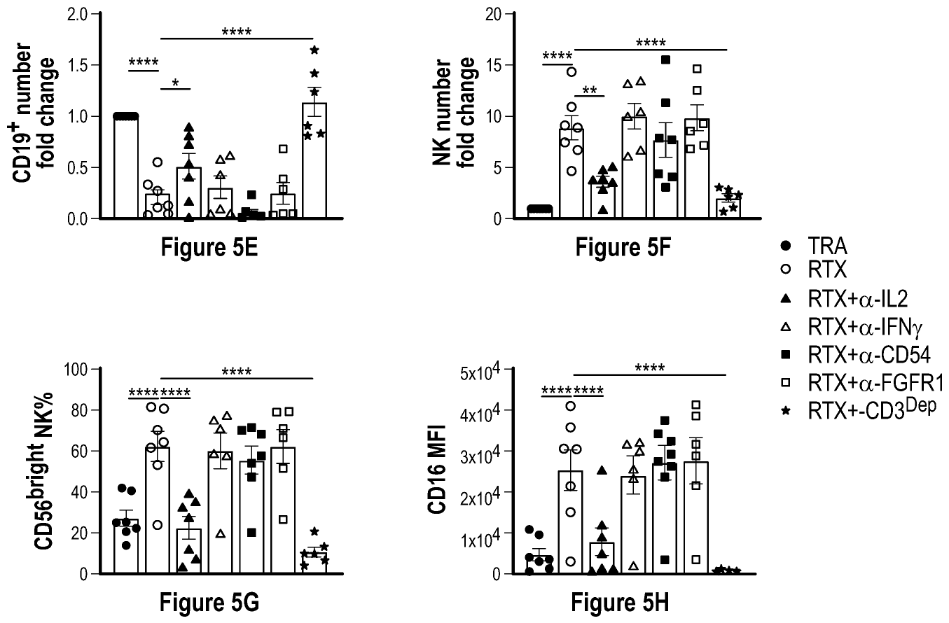

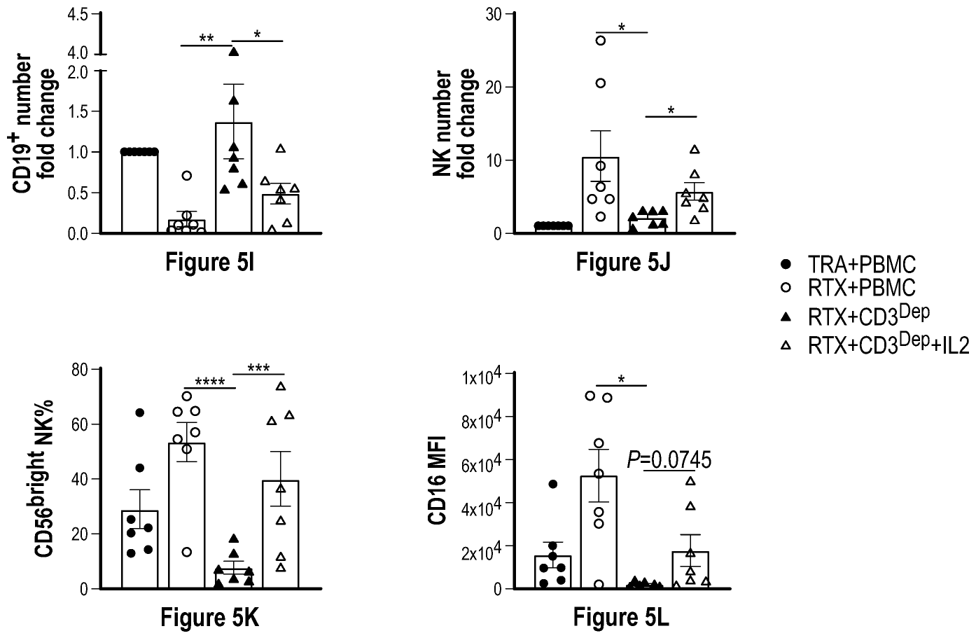

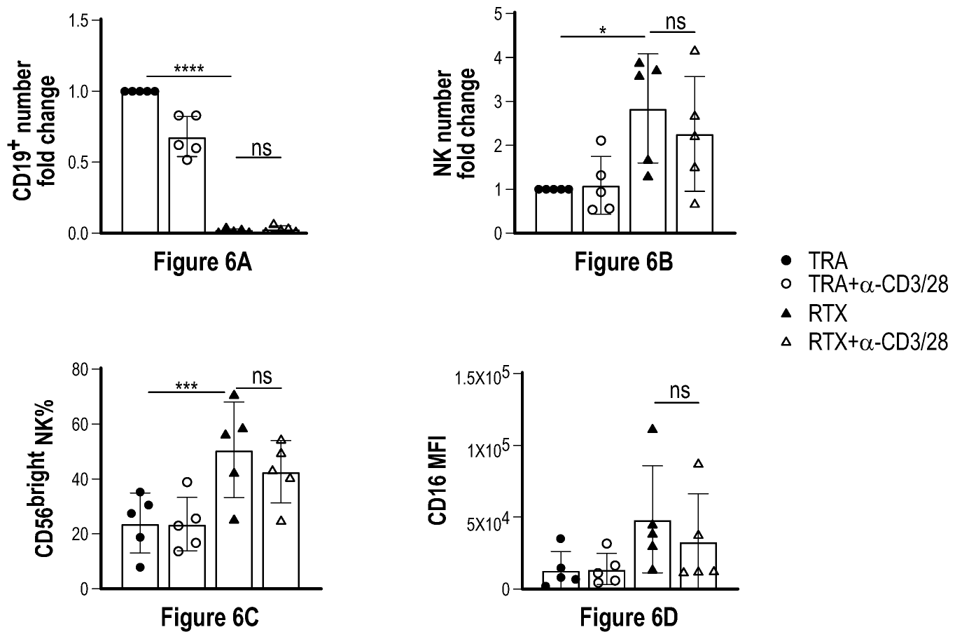

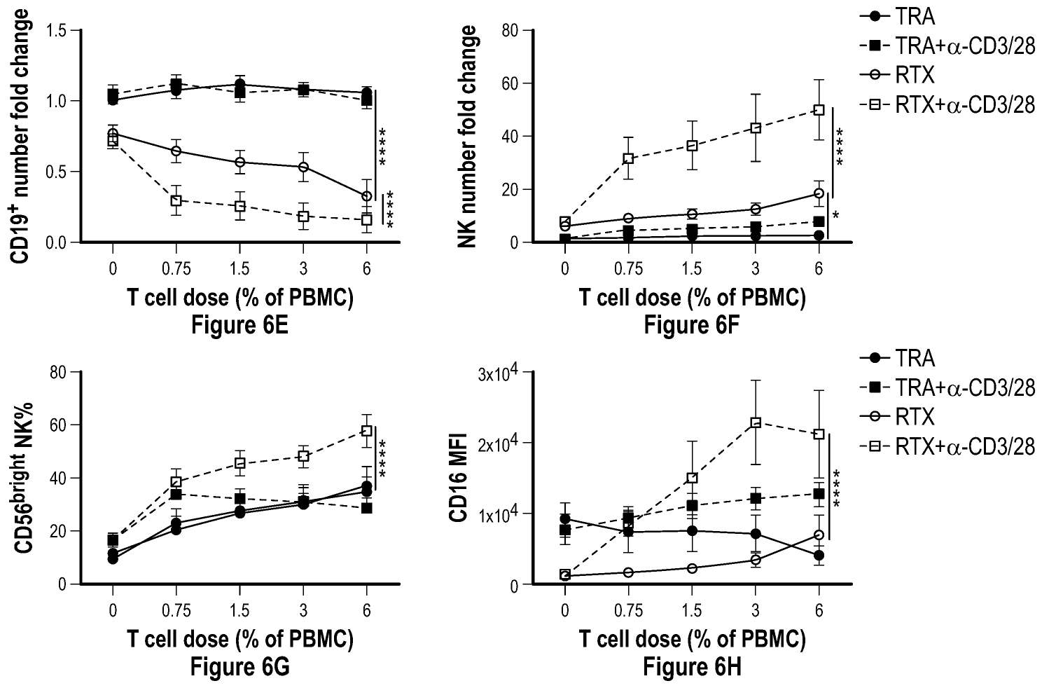

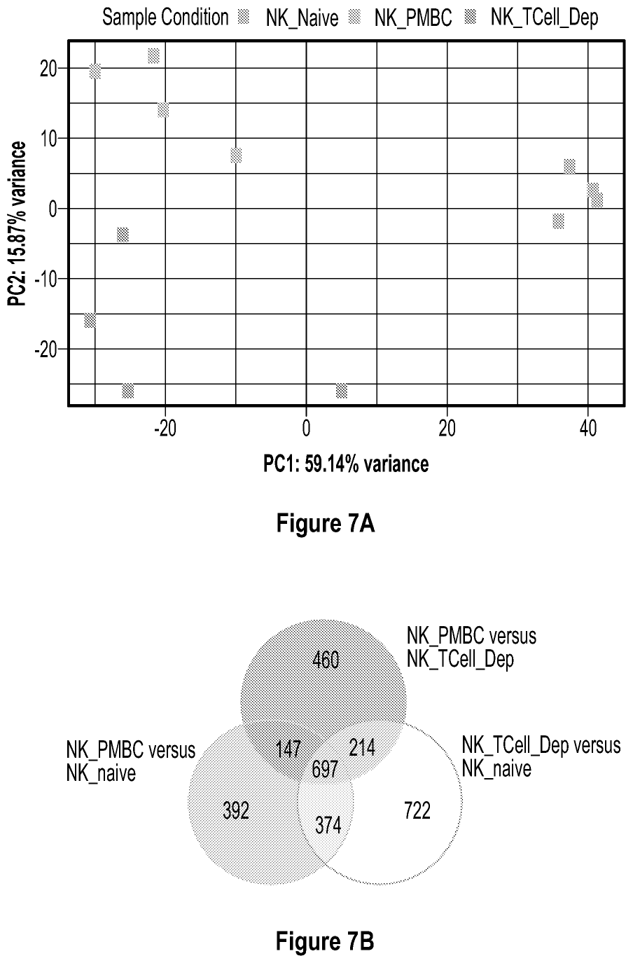

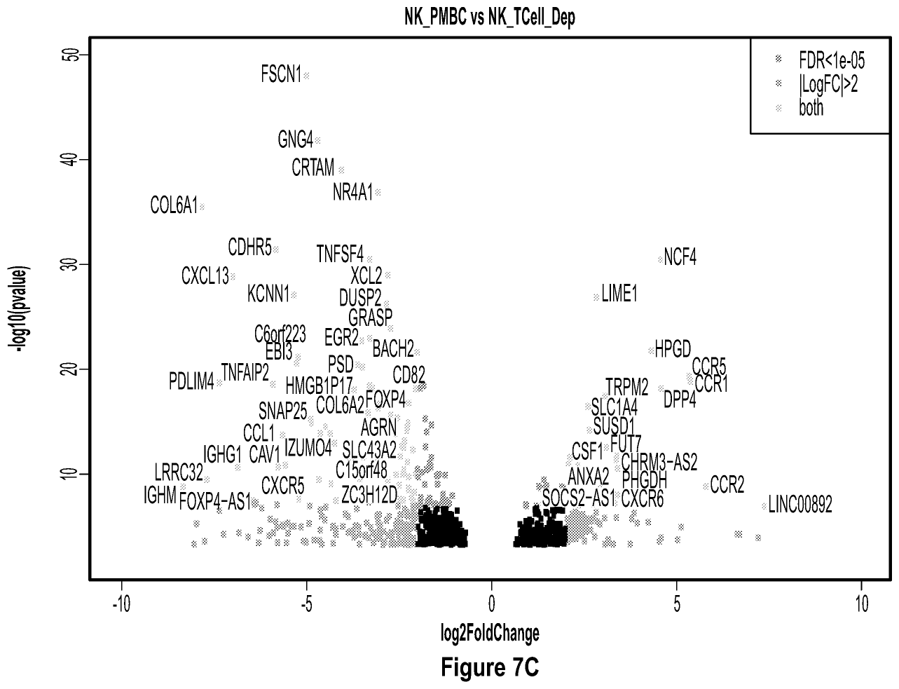

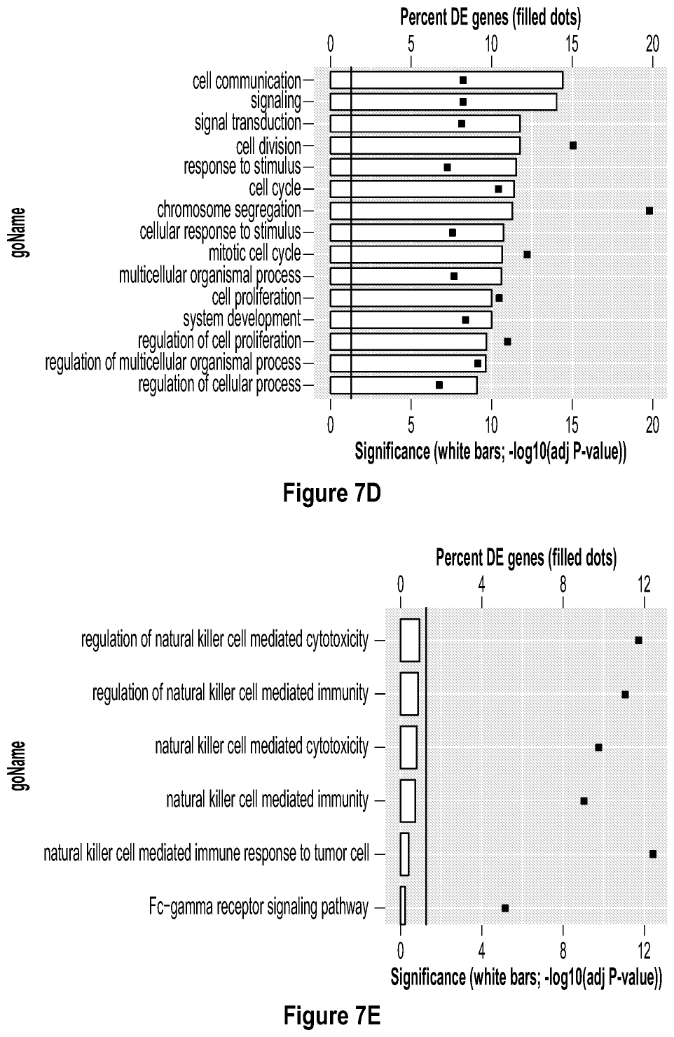

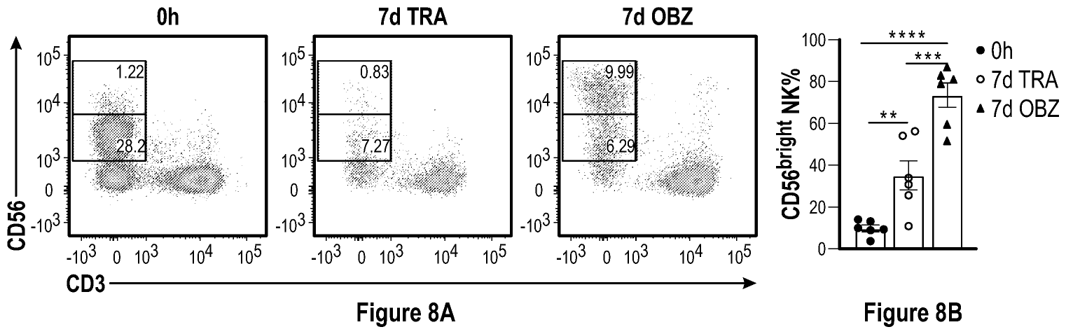

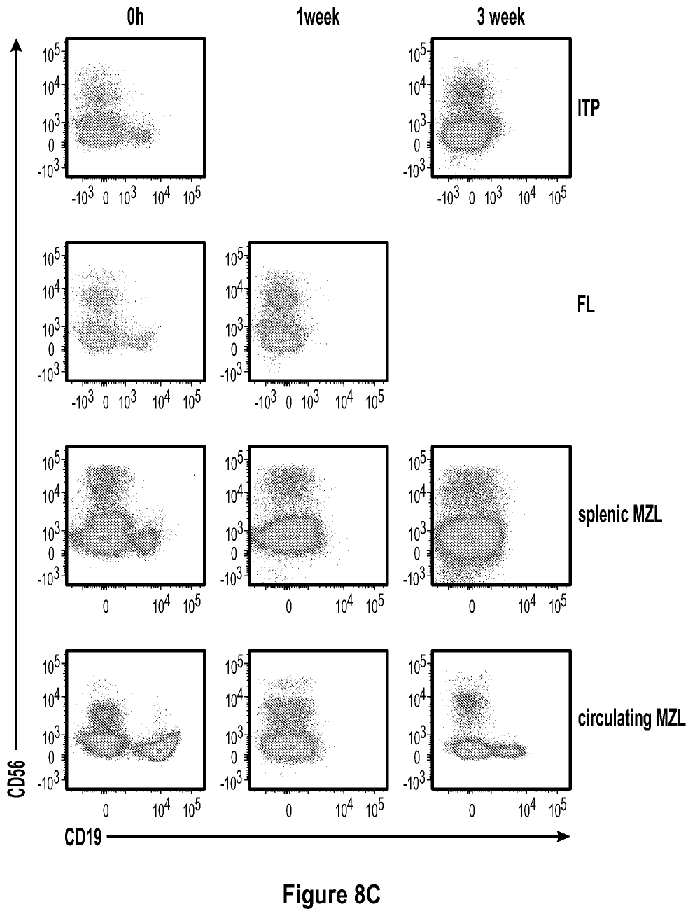

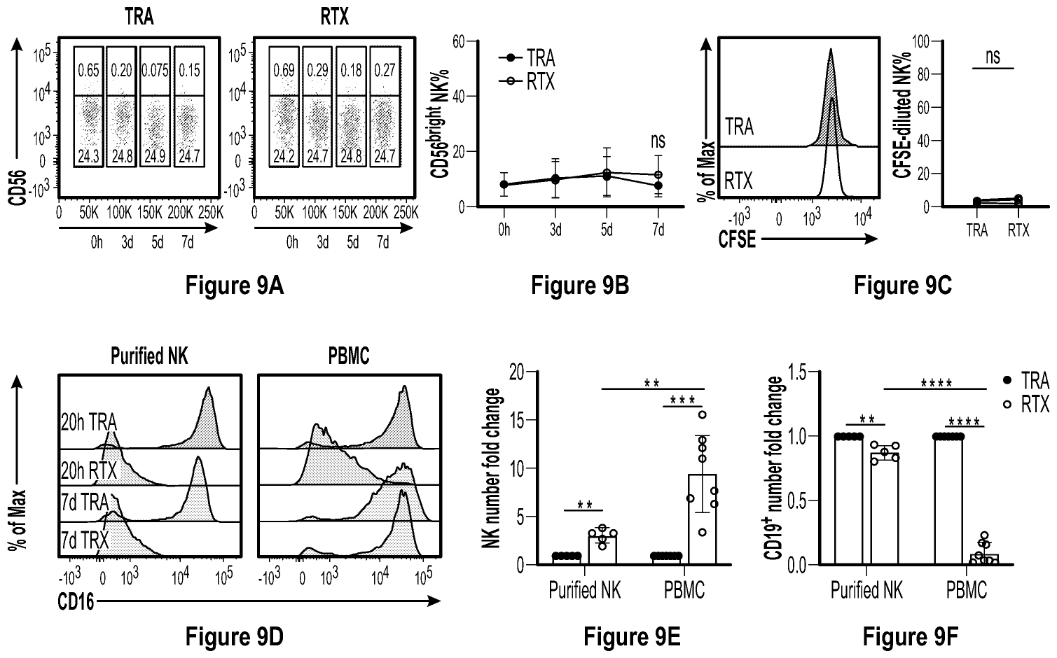

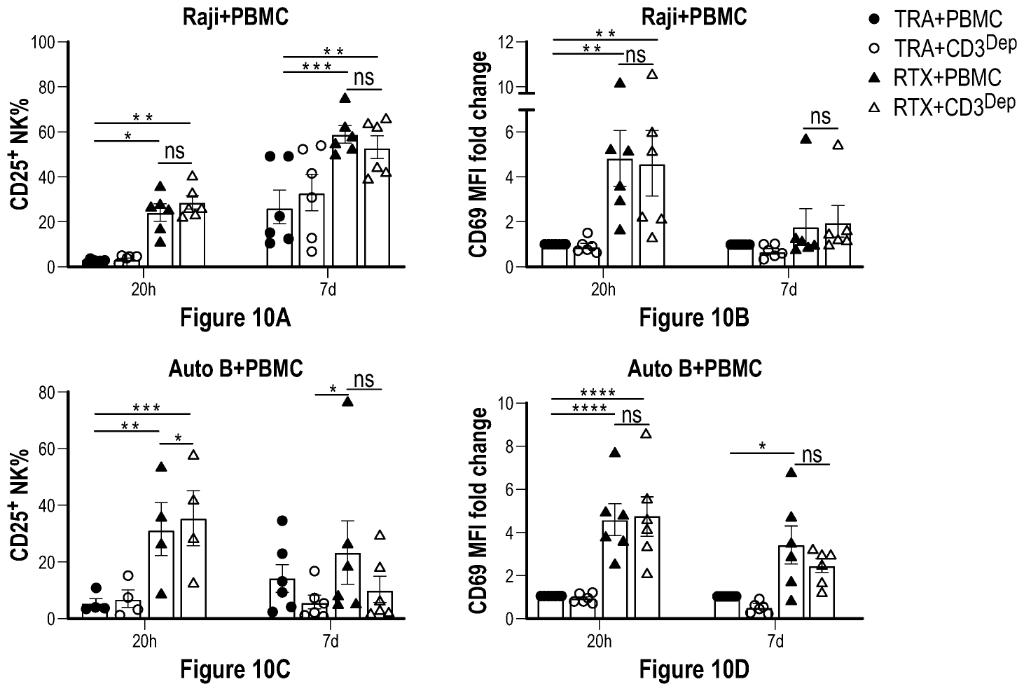

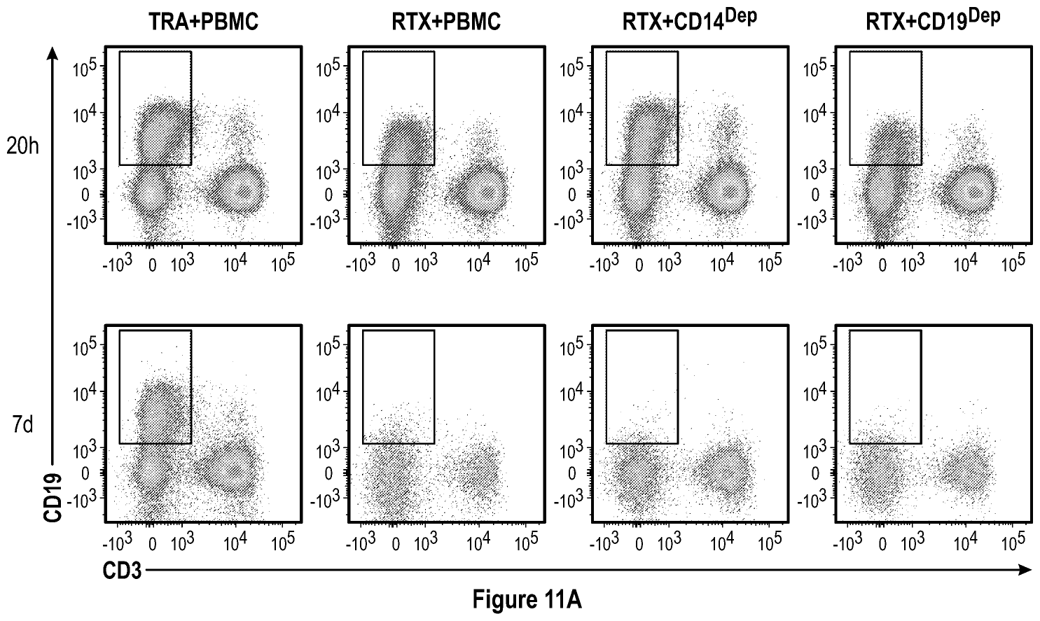

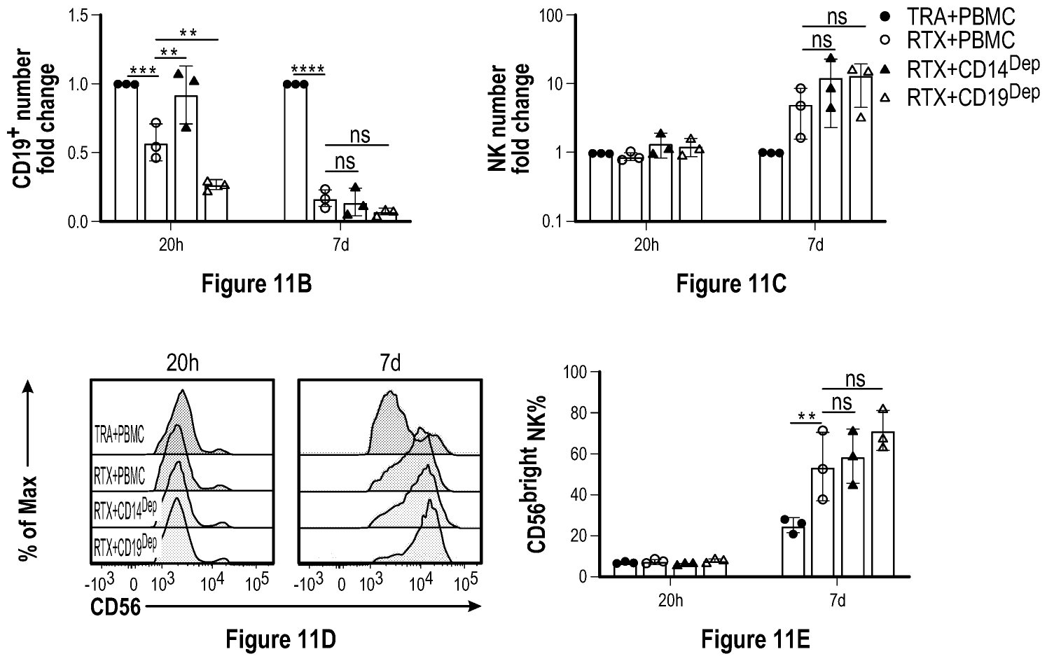

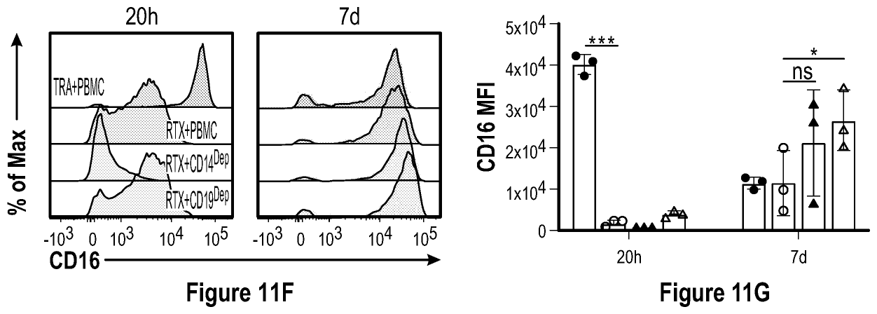

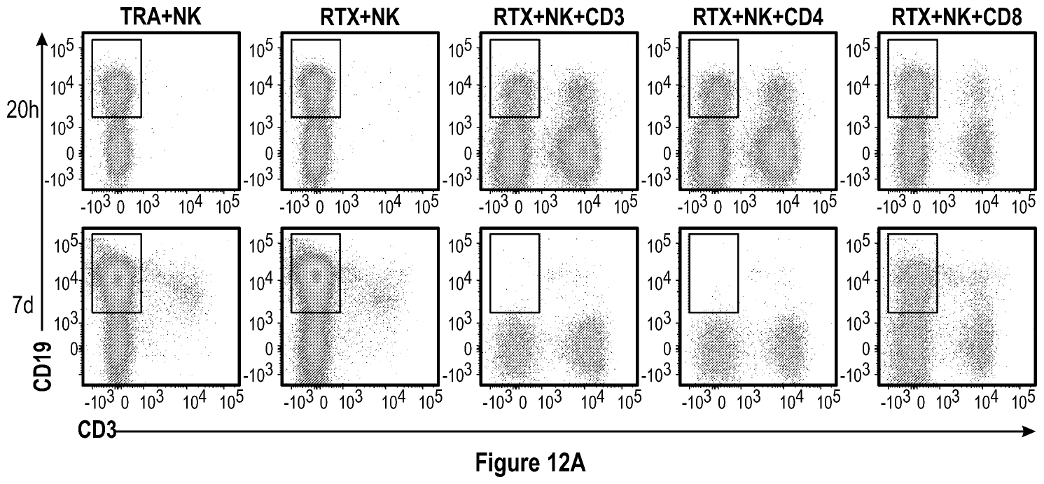

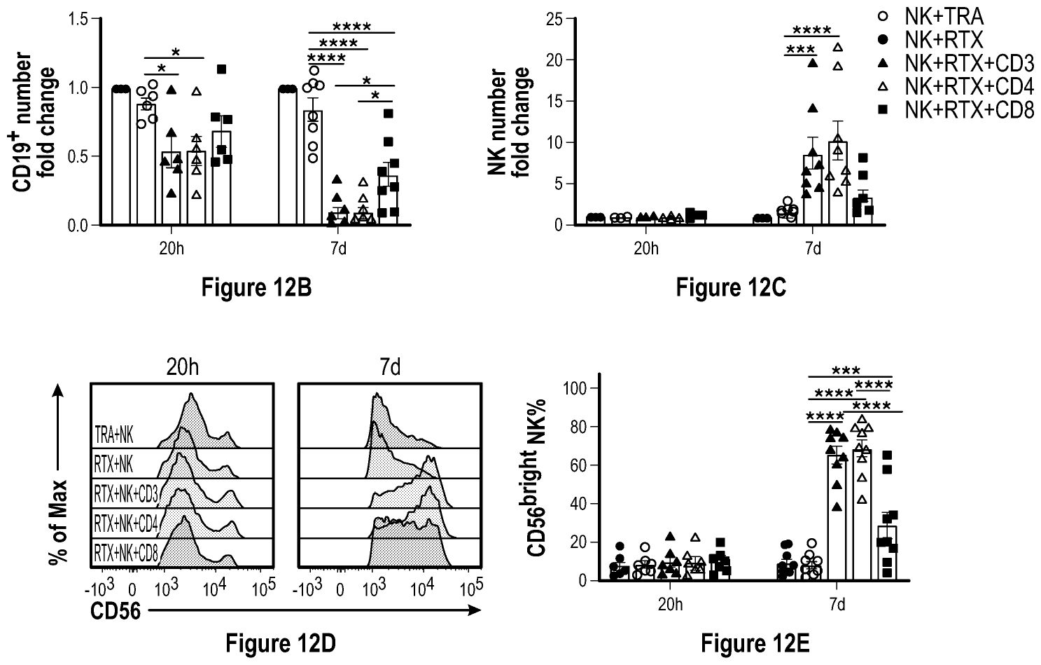

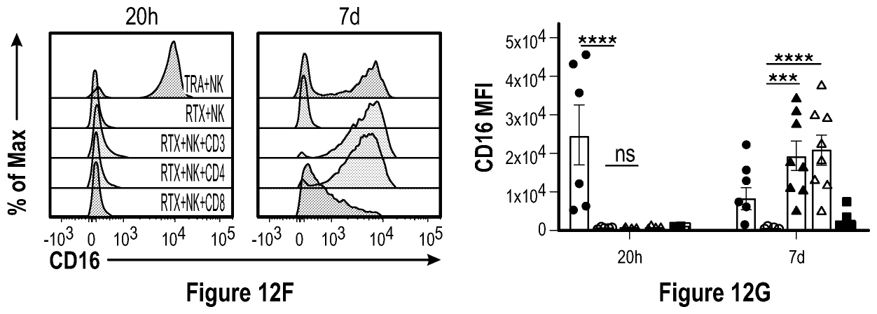

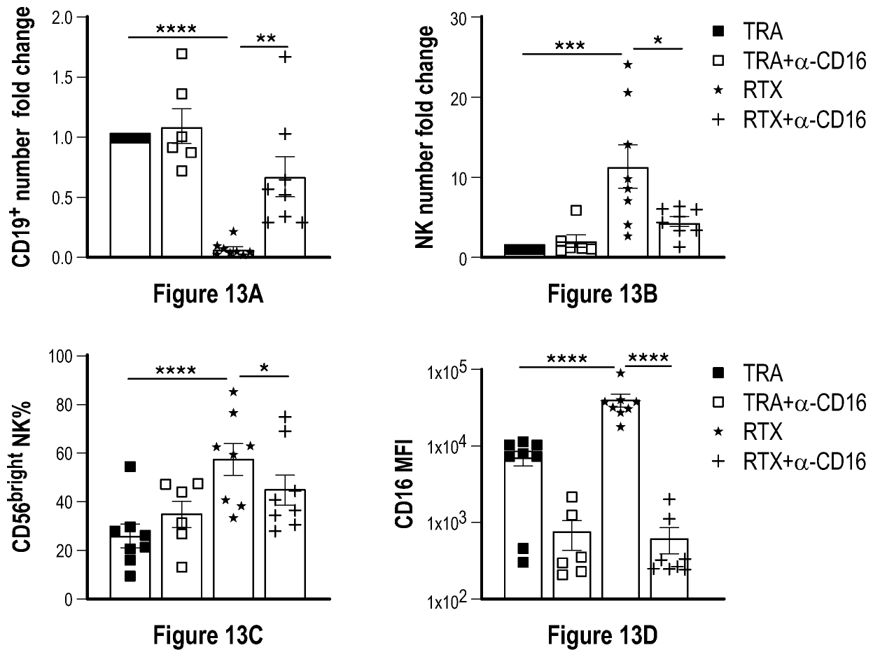

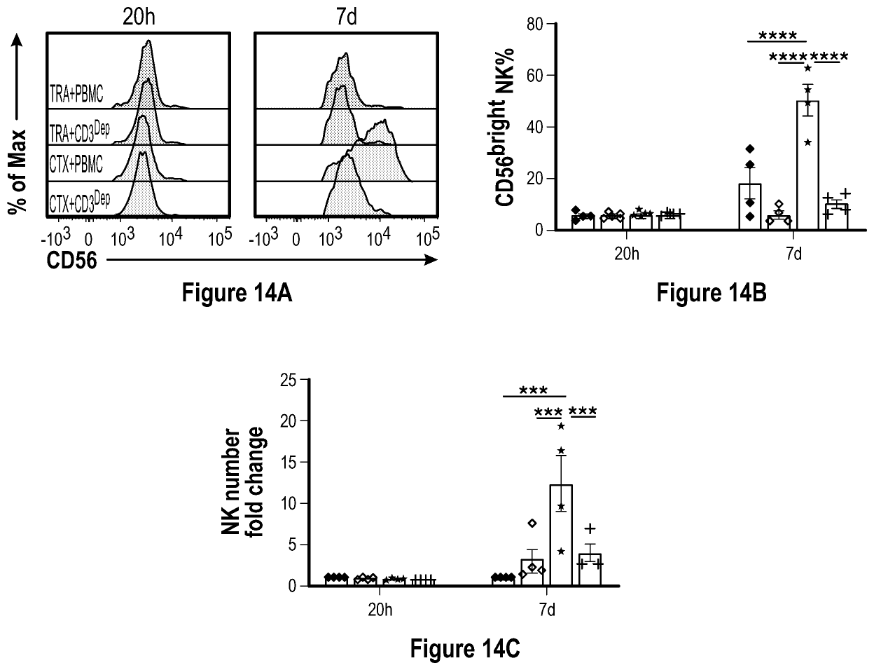

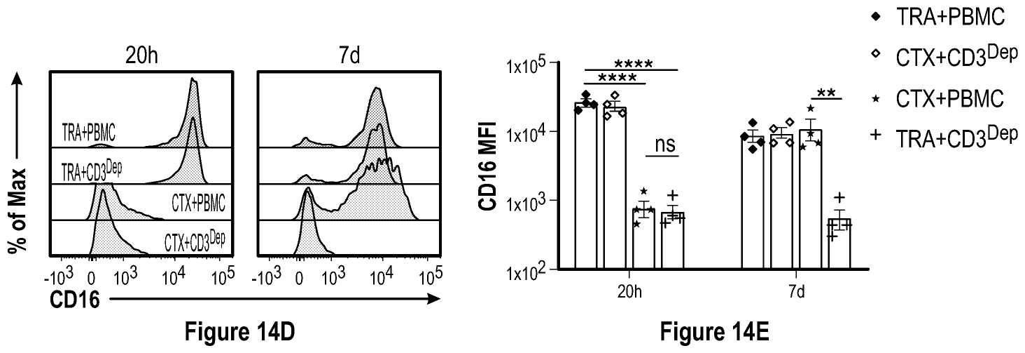

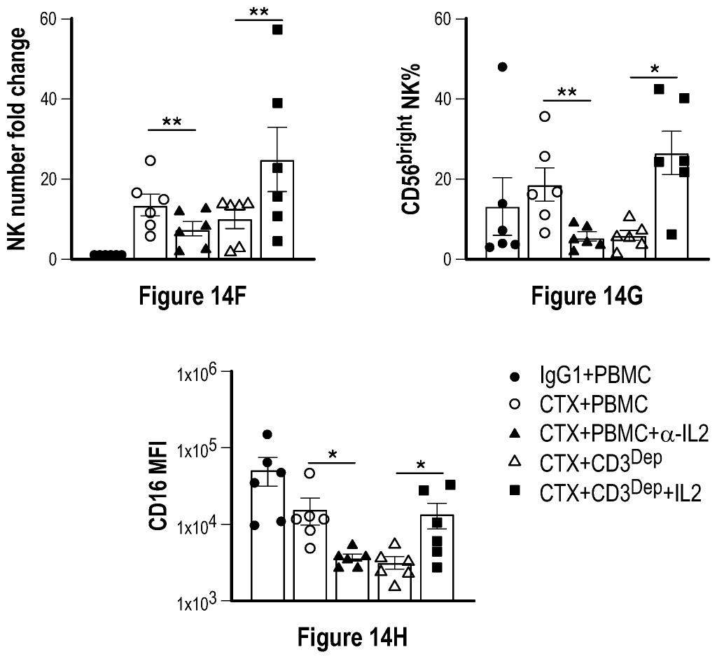

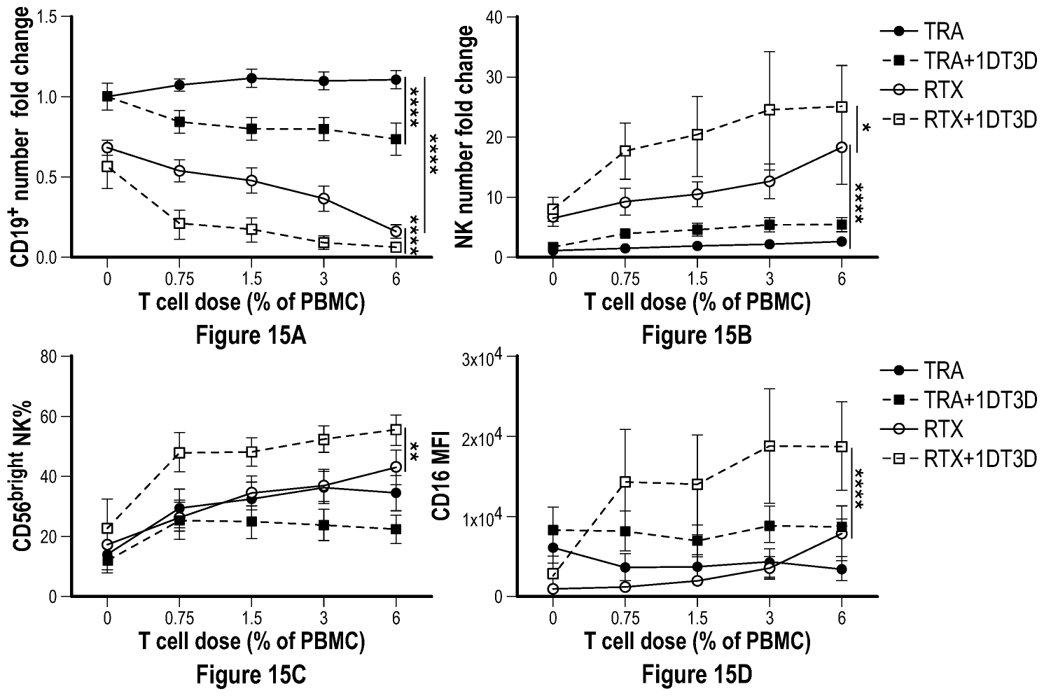

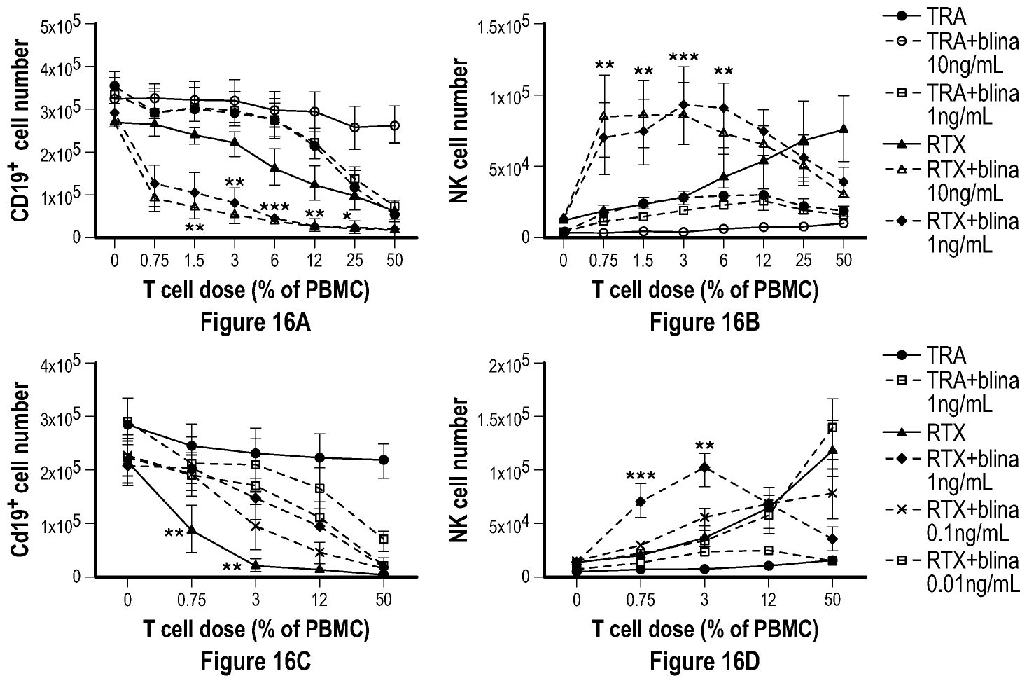

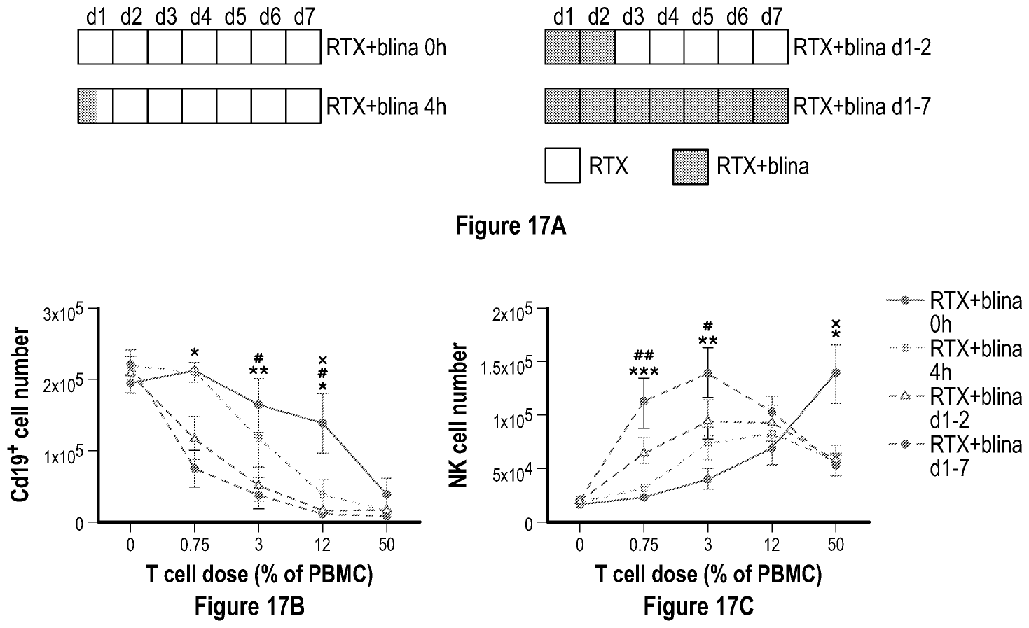

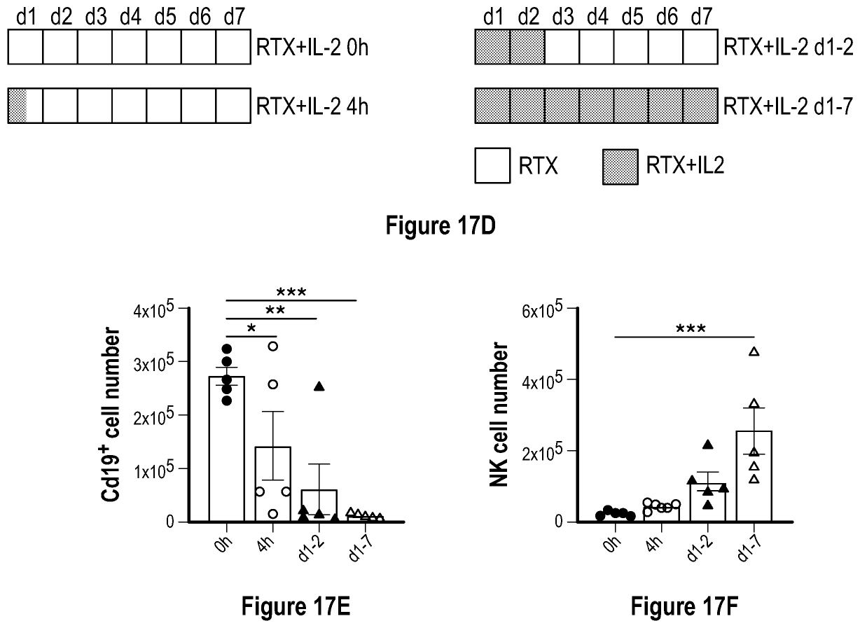

Unfractionated PBMCs were cocultured with Raji cells and RTX or TRA for 7 days. a-IL2, a-IFNg, a-CD54, or a-FGFR1 mAb (10 ug/ml) was added to the coculture. (FIGS. 5E-5H) On day 7, IL2 neutralization suppressed RTX-mediated CD19⁺ target cell elimination, NK viability, CD56^{dim} to CD₅₆^{bright} NK transition and CD16 re-expression by NK cells. n=6-7 Unfractionated PBMC or PBMC depleted of CD3⁺ T cells were cocultured with Raji cells and RTX or TRA for 7 days. Recombinant IL2 (20 ng/ml) was added to the coculture. (FIGS. 5I-5L) On day 7, IL2 supplementation increased RTX-mediated cytotoxicity, viability, CD56^{dim} to CD56^{bright} transition and CD16 re-expression of NK cells in T cell-depleted PBMCs. n=7. Cell counts in the TRA group were used to normalize cell numbers. Dep: depleted.

FIGS. 6A-6H. T cell activation enhances RTX-mediated NK cell responses. PBMC were cocultured with Raji cells and RTX or TRA for 7 days. Anti-CD3/28 beads were added to the coculture to activate T cells. (FIGS. 6A-6D) RTX-mediated NK cell cytotoxicity, NK cell persistence, CD56^{dim} to CD₅₆^{bright} transition and CD16 recovery is not altered by anti-CD3/28 mediated T cell activation. Cell counts in the TRA group were used to normalize cell numbers. n=5.

PBMC depleted of CD3⁺ T cells were cocultured with Raji cells and RTX or TRA for 7 days. Serial dilutions of either autologous resting or anti-CD3/28 activated T cells (from 0.75% to 12% of the PBMC amount) were added to the coculture. (FIGS. 6E-6H) RTX-mediated NK cell cytotoxicity, persistence, CD56^{dim} to CD₅₆^{bright} transition and CD16 re-expression is T cell dose dependent and further enhanced by T cell activation. Cell counts in the TRA group at 0% T cell dose were used to normalize cell numbers. n=7.

FIGS. 7A-7E. T cells impact RTX-mediated NK cell transcriptomics.

(FIG. 7A) The transcriptomics of NK cells isolated from different experimental conditions: NK_naive, NK_PBMC and NK_TCell_Dep were easily separated by PCA. (FIG. 7B) Summary of DEGs from three conditions. (FIG. 7C) NK_PBMC versus NK_TCell_Dep volcano plot of DEG. (FIG. 7D) Top biological processes enriched by DEGs of NK_PBMC versus NK_TCell_Dep indicating the presence of T cells enhanced expression of genes involved in cell communication, response to stimulation, cell division, cell cycle, chromosome segregation and cell proliferation. (FIG. 7E) Depletion of T cells does not impact the biological pathways involved in NK cell cytotoxicity and Fcg receptor signaling at the transcriptional level.

FIGS. 8A-8C. CD56^{dim} to CD56^{bright} NK cell phenotypical change in response to OBZ and in patients receiving RTX infusion. (FIGS. 8A, 8B) PBMC were cocultured with Raji cells and OBZ or TRA for 7 days. OBZ induces enhanced expression of CD56 on NK cells. n=6. (FIG. 8C) Patients were treated by weekly single agent RTX infusion. CD19⁺ target cells were eliminated 1 week after RTX treatment, but re-emerged on week 3 in the patient with circulating tumors.

FIGS. 9A-9F. RTX fails to induce CD56^{dim} to CD56^{bright} transition and proliferation of isolated NK cells. Isolated NK cells or unfractionated PBMCs were cocultured with Raji cells and RTX or TRA for up to 7 days. (FIGS. 9A, 9B) RTX has no impact on CD56 expression by isolated NK cells. n=4. (FIG. 9C) RTX fails to induce CFSE dilution by isolated NK cells at 7 days. n=5. (FIG. 9D) CD16 re-expression is seen with unfractionated PBMCs, but not with isolated PBMCs, after culture for 7 days with RTX. (FIG. 9E) The number of NK cells remaining after 7-day culture with RTX is greater with unfractionated PBMCs compared to isolated NK cells. (FIG. 9F) Elimination of CD19⁺ target cells after a 7-day culture is greater with unfractionated PBMCs compared to isolated NK cells. n=5-8. Cell counts in the TRA group were used to normalize cell numbers.

FIGS. 10A-10D. T cell depletion does not impact RTX-mediated NK cell activation. Unfractionated PBMC or PBMC depleted of CD3⁺ cells were cocultured with Raji or autologous B cells and RTX or TRA for 7 days. Depletion of T cells does not alter RTX-activated NK expression of CD25 (FIGS. 10A, 10C) or CD69 (FIGS. 10B, 10D). n=4-6.

FIGS. 11A-11G. The depletion of CD14+ monocytes or CD19+ B cells does not suppress RTX-mediated NK cell responses. Unfractionated PBMC or PBMC depleted of CD14′ monocytes or CD19⁺ normal B cells were cocultured with Raji cells and RTX or TRA for 7 days and elimination of CD19⁺ target cells determined by flow cytometry. (FIGS. 11A, 111B) The depletion of monocytes or B cells does not impact RTX-mediated NK elimination of CD19⁺ cells. (FIG. 11C) The depletion of monocytes or B cells does not impact on RTX-mediated NK cell viability. (FIGS. 11D, 11E) The depletion of monocytes or B cells does not impact on RTX-mediated CD56dim to CD56bright NK cell transition. (FIGS. 11F, 11G) The depletion of monocytes or B cells does not suppress RTX-mediated recovery of CD16. n=3.

FIGS. 12A-12G. T cells, mainly CD4⁺ cells, are essential for RTX-mediated NK cell responses. Isolated NK cells were cocultured with Raji cells and RTX or TRA. T cell subsets were added to the culture based on their physiological proportion in the peripheral blood: 0.6 million CD3*, 0.4 million CD4⁺, or 0.2 million CD8⁺ T cells. (FIGS. 12A, 12B) The elimination of CD19⁺ target cells is significantly enhanced with the presence of CD3⁺, CD4⁺ or CD8⁺ T cells at day 7. CD3⁺ and CD4⁺ T cells improve elimination of CD19⁺ target cells to a greater degree than CD8⁺ T cells. (FIG. 12C) The number of NK cells remaining in the culture after 7 days is increased by the addition of CD3⁺ or CD4⁺ T cells but not by CD8⁺ T cells. (FIGS. 12D, 12E) CD56^{dim} to CD₅₆^{bright} NK transition is only seen when T cells were present. CD3⁺ and CD4⁺ induce greater CD56^{dim} to CD56^{bright} NK transition than CD8⁺ T cells. (FIGS. 12F, 12G) CD16 re-expression at 7 days is only seen when CD3⁺ or CD4⁺ T cells are present. n=6-8. Cell counts in the TRA group were used to normalize cell numbers.

FIGS. 13A-13D. CD16 blockade inhibits RTX-mediated NK cell responses. PBMCs were cocultured with Raji cells and RTX or TRA for 7 days. Polyclonal anti-CD16 neutralization Ab (10 μg/ml) was added to block the CD16 signaling. On day 7, the elimination of CD19⁺ target cells (FIG. 13A), the number of NK cells (FIG. 13B), and CD56dim to CD56^{bright} transition (FIG. 13C) is inhibited by anti-CD16 Ab. The expression of CD16 (FIG. 13D) on NK cells is minimally detectable in the presence of anti-CD16. n=6-8.

FIGS. 14A-14H. T cells maintain CTX-mediated NK cell responses via IL2. Unfractionated PBMCs or PBMCs depleted of CD3⁺ T cells were cocultured with SQ20B cells and CTX or TRA. On day 7, CTX induces CD56^{dim} to CD56^{bright} NK cell transition (FIGS. 14A, 14B), maintains NK cell viability (FIG. 14C), and CD16 re-expression by NK cells after the intial downregulation at 20 hours (FIGS. 14D, 14E) in unfractionated PBMCs but not in T cell-depleted PBMCs. n=4.

(FIGS. 14F-14H) Unfractionated PBMCs or PBMCs depleted of CD3⁺ T cells were cocultured with SQ20B cells and CTX or IgG1 control. α-IL2 blocking mAb (10 μg/ml) or recombinant IL2 (20 ng/ml) was added to the coculture for 7 days. On day 7, CTX-mediated NK cell viability, CD56^{dim} to CD₅₆^{bright} NK transition, and CD16 re-expression on NK cells in unfractionated PBMCs were suppressed by a-IL2, and was maintained by recombinant IL2 supplementation on NK cells in T cell-depleted PBMCs. Cell counts in the TRA+PBMC group are used to normalize cell numbers. n=6.

FIGS. 15A-15D. T cell activation by 1DT3D enhances RTX-mediated NK cell responses. PBMC depleted of CD3⁺ T cells were cocultured with Raji cells and RTX or TRA for 7 days. Serial dilutions of either autologous resting or 1DT3D-activated T cells were added to the coculture. RTX-mediated NK cell elimination of target cells (FIG. 15A), persistence (FIG. 15B), CD56^{dim} to CD56^{bright} transition (FIG. 15C) and CD16 re-expression (FIG. 15D) is T cell dose dependent and further enhanced by 1DT3D-mediated T cell activation. n=5. Cell counts in the TRA group at 0% T cell dose were used to normalize cell numbers.

FIGS. 16A-16D. Blinatumomab enhances RTX-mediated NK cell response. PBMCs depleted of CD3⁺ T cells were cocultured with Raji cells and RTX or trastuzumab (TRA) as the control for 7 days. Serial dilutions (from 0.75 to 50% of PBMCs) of autologous CD3⁺ T cells were added back as was blinatumomab to select samples. NK cell response was measured on day 7. (FIGS. 16A, 16B). RTX-mediated NK cell elimination of CD19⁺ target cells and NK cell viability increase in a T cell dose-dependent manner. These changes are enhanced by blinatumomab (1 ng/mL or 10 ng/mL) at low T cell percent from 0.75 to 6%. n=6. (FIGS. 16C, 16D). Lower concentrations of blinatumomab at 0.1 ng/mL or 0.01 ng/mL minimally impact on RTX-mediated NK cell ADCC or viability. n=5. Student's t test was used to calculate statistical significance. *p<0.05; **p<0.01; ***p<0.001 indicate RTX versus RTX+blina 10 ng/mL. blina blinatumomab.

FIGS. 17A-17F. Short-term blinatumomab exposure enhances NK cell ADCC and viability. PBMCs depleted of T cells were cocultured with Raji cells and RTX for 7 days. Serial dilutions (from 0.75 to 50% of PBMCs) of autologous T cells were added back. (FIG. 17A). Blinatumomab (1 ng/mL) was supplemented for the first 4 h (4 h), 2 days (d1-2), 7 days (d1-7) or not added (0 h). After the indicated time, blinatumomab was washed out and the coculture was refreshed with RTX-containing medium. (FIGS. 17B, 17C). 2-day blinatumomab enhances RTX-mediated NK cell killing of CD19*target cells and viability. 4-h blinatumomab enhances NK cell ADCC at 12% T cells but fails to increase NK cell viability. n=6. Student's t test was used to calculate statistical significance. *p<0.05; **p<0.01; ***p<0.001 are the comparisons between RTX+blina d1-7 versus RTX+blina 0 h; #p<0.05, ##p<0.01 indicate RTX+blina d1-2 versus RTX+blina 0 h; ×p<0.05 indicates RTX+blina 4 h versus RTX+blina 0 h. (FIG. 17D). PBMCs depleted of T cells were cocultured with Raji cells and RTX for 7 days. Recombinant TL-2 (20 ng/mL) was supplemented for 4 h (4 h), 2 days (d1-2), 7 days (d1-7) or not added (0 h). TL-2 was washed out and replaced by RTX-containing medium after the indicated time. (FIGS. 17E, 17F). 2-day or 4-h TL-2 exposure enhances NK cell ADCC of CD19⁺ target cells. Short-term TL-2 treatment also increases the number of viable NK cells, although not statistically significant. n=5. One-way ANOVA was used to calculate statistical significance. *p<0.05; **p<0.01; ***p<0.001.

### DETAILED DESCRIPTION EXEMPLARY EMBODIMENTS AND DEFINITIONS

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The term “about” means±10%.

The terms “treat” and “treatment” refer to both therapeutic treatment and prophylactic or preventative measures, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (NSCLC), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Gastric cancer, as used herein, includes stomach cancer, which can develop in any part of the stomach and may spread throughout the stomach and to other organs, particularly the esophagus, lungs, lymph nodes, and the liver.

A “chemotherapeutic agent” is a biological (large molecule) or chemical (small molecule) compound useful in the treatment of cancer, regardless of mechanism of action. Classes of chemotherapeutic agents include, but are not limited to alkylating agents, antimetabolites, spindle poison plant alkaloids, cytotoxic/antitumor antibiotics, topoisomerase inhibitors, proteins, antibodies, photosensitizers, and kinase inhibitors. Chemotherapeutic agents include compounds used in “targeted therapy” and non-targeted conventional chemotherapy.

The term “mammal” includes, but is not limited to, humans, mice, rats, guinea pigs, monkeys, dogs, cats, horses, cows, pigs, sheep, and poultry.

The term “package insert” is used to refer to instructions customarily included in commercial packages of therapeutic products, that contain information about the indications, usage, dosage, administration, contraindications and/or warnings concerning the use of such therapeutic products.

The phrase “pharmaceutically acceptable” indicates that the substance or composition must be compatible chemically and/or toxicologically, with the other ingredients comprising a formulation, and/or the mammal being treated therewith.

The term “synergistic” as used herein refers to a therapeutic combination which is more effective than the additive effects of the two or more single agents. A determination of a synergistic interaction between a T cell activating agent and one or more chemotherapeutic agent may be based on the results obtained from the assays described herein. The results of these assays can be analyzed using the Chou and Talalay combination method and Dose-Effect Analysis with CalcuSyn software in order to obtain a Combination Index (Chou and Talalay, 1984, Adv. Enzyme Regul. 22:27-55). The combinations provided by this invention have been evaluated in several assay systems, and the data can be analyzed utilizing a standard program for quantifying synergism, additivism, and antagonism among anticancer agents. The program utilized is that described by Chou and Talalay, in “New Avenues in Developmental Cancer Chemotherapy,” Academic Press, 1987, Chapter 2. Combination Index values less than 0.8 indicates synergy, values greater than 1.2 indicate antagonism and values between 0.8 to 1.2 indicate additive effects. The combination therapy may provide “synergy” and prove “synergistic,” i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together. In some examples, Combination effects were evaluated using both the BLISS independence model and the highest single agent (HSA) model (Lehir et al. 2007, Molecular Systems Biology 3:80). BLISS scores quantify degree of potentiation from single agents and a BLISS score >0 suggests greater than simple additivity. An HSA score >0 suggests a combination effect greater than the maximum of the single agent responses at corresponding concentrations.

### Methods of Treatment

A major mechanism of action of anticancer monoclonal antibodies that bind to antigens on cancer cells (rituximab, cetuximab, etc.) is NK cell mediated antibody dependent cellular cytotoxicity (ADCC). It is well known that NK cells are activated by antibody-coated cancer cells and then mediate ADCC. The inventors unexpectedly found that T cells, mainly CD4⁺, enhance the NK cell activation phenotype in the longer term, and are critical to maintaining the viability of NK cells activated by antibody-coated cancer cells. Thus, T cells enhance the ability of NK cells to mediate ADCC and kill cancer cells. This effect is partially, but not totally mediated by local production of IL2 by CD4⁺ cells. Importantly, it was found that activation of T cells by anti-CD3×anti-cancer bispecific antibodies in the presence of cancer significantly enhanced the ability of T cells to support NK cell viability, and this, in turn enhanced ADCC. Enhanced ADCC mediated by anti-CD3×anti-cancer bispecific antibodies was seen with very small numbers of T cells, very low concentrations of bispecific antibody, and short exposure to bispecific antibody. This unexpected finding suggests intermittent therapy with bispecific anti-CD3×anti-tumor antibody, given along with monospecific anti-tumor antibodies could result in improved anti-cancer activity. This mechanism of action is distinct from retargeting of T cells to kill tumor cells that is the currently the basis for use of anti-CD3×anti-cancer bispecific antibodies as an anti-cancer therapy.

Clinically, this finding suggests the addition of bispecific anti-CD3×anti-tumor antibody, delivered as a bolus intravenously, subcutaneously, or by a short infusion, could enhance the efficacy of a broad variety of monospecific anti-cancer antibodies if the two are administered simultaneously or in a manner where they are both present in the tumor microenvironment at the same time. No known studies have been done exploring simultaneous cancer therapy involving anti-cancer monospecific antibody and anti-CD3×anti-cancer bispecific antibodies. Based on this newly discovered mechanism of action, anti-CD3×anti-cancer bispecific antibodies enhance the efficacy of other NK-based anti-cancer therapies including adoptively transferred NK cells (unmodified or genetically altered) or bispecific anti-NK×anti-cancer antibodies.

In one aspect, a method is provided for treating a hyperproliferative disorder in a mammal in need thereof comprising, administering to the mammal a combination of (a) an anti-cancer antibody and (b) a T cell activating agent for the prophylactic or therapeutic treatment of the hyperproliferative disorder.

In certain aspects, the hyperproliferative disorder to be treated is cancer. In certain aspects, the cancer is B cell Lymphoma, T cell Lymphoma, Myeloma, Non-small cell lung cancer, Small cell lung cancer, Breast cancer, Head and neck cancer, Neuroblastoma, Soft tissue sarcoma, Gastric cancer, Colorectal cancer, Chronic lymphocytic leukemia, Acute lymphoblastic leukemia, Chronic myeloid leukemia, Acute myeloid leukemia, Pancreatic cancer, or Prostate cancer.

In one aspect the invention provides a method for treating a hyperproliferative disorder in a mammal in need thereof, wherein administration of an anti-cancer antibody with an T cell activating agent, provides a synergistic effect in treating the hyperproliferative disorder. In a further aspect, the synergistic effect has a Combination Index value of less than about 0.8.

In one aspect, anti-cancer antibody is administered in combination with an T cell activating agent to treat cancer.

In certain aspects, a method is provided for maintaining long-term natural killer (NK) cell antibody-dependent cellular cytotoxicity (ADCC) in the treatment of a hyperproliferative disorder in a mammal in need thereof comprising, administering to the mammal a combination of (a) an anti-cancer antibody, and (b) a T cell activating agent for the prophylactic or therapeutic treatment of the hyperproliferative disorder.

In certain aspects, the anti-cancer antibody and the T cell activating agent are administered separately, simultaneously or sequentially.

In certain aspects, the anti-cancer antibody and the T cell activating agent are administered simultaneously.

In certain aspects, the administration is repeated weekly.

In certain aspects, the administration is repeated monthly.

In certain aspects, the administration is repeated for up to six months, for example, for one month, two months, three monthly, four months, five months or six months.

In certain aspects, the T cell activating agent is administered intravenously or subcutaneously.

In certain aspects, the bispecific antibody is administered at a dose of between 2 and 50 micrograms. In certain aspects, the dose is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 micrograms.

In certain aspects, a method is provided for a use of a T cell activating agent, for therapeutic use for improving the quality of life of a patient treated for a hyperproliferative disorder with an anti-cancer antibody.

In certain aspects, a method is provided for a use of a T cell activating agent in the preparation of a medicament for the treatment of a hyperproliferative disorder in a mammal treated with an anti-cancer antibody, wherein the T cell activating agent is administered to the mammal.

In certain aspects, a kit is provided comprising a T cell activating agent, a container, and a package insert or label indicating the administration of the T cell activating agent with an anti-cancer antibody for treating a hyperproliferative disorder.

In certain aspects, a product is provided comprising an anti-cancer antibody and an T cell activating agent as a combined preparation for separate, simultaneous, or sequential use in the treatment of a hyperproliferative disorder.

T Cell Activating Agents Certain T cell activating agents have demonstrated surprising and unexpected properties in combination with anti-cancer antibodies in inhibiting cellular proliferation in vitro and in vivo.

In certain aspects, the T cell activating agent activates CD4+ T cells. In certain aspects, the T cell activating agent is a bispecific antibody, a chimeric antigen receptor T (CAR-T) cells, and/or a vaccine that induces a T cell response. In certain aspects, the T cell activating agent is a bispecific antibody. In certain aspects, the T cell activating agent is a bispecific anti-CD3×anti-tumor antibody. In certain aspects, the bispecific anti-CD3×anti-tumor antibody is Blinatumomab or Catumaxomab. In certain aspects, the bispecific anti-CD3×anti-tumor antibody is Blinatumomab. In certain aspects, the bispecific anti-CD3×anti-tumor antibody is Catumaxomab.

### Anti-Cancer Antibodies

In certain aspects, the anti-cancer antibody mediates antibody-dependent cellular cytotoxicity (ADCC). In certain aspects, the ADCC is mediated by natural killer (NK) cells. In certain aspects, the anti-cancer antibody is a monospecific antibody. In certain aspects, the monospecific antibody is a monoclonal antibody. There are many monoclonal antibodies that are approved by the FDA or are in advanced clinical trials that can be combined with bispecific anti-CD3×anti-cancer antibodies based on this mechanism. These include the following:

| | | | |
|---|---|---|---|
| Margetuximab | MARGENZA | HER2; Chimeric IgG1 | HER2+ breast cancer |
| Naxitamab | DANYELZA | GD2; Humanized IgG1 | High-risk neuroblastoma and |
| | | | refractory osteomedullary disease |
| Tafasitamab | Monjuvi | CD19; Humanized IgG1 | Diffuse large B-cell lymphoma |
| Isatuximab | Sarclisa | CD38; Chimeric IgG1 | Multiple myeloma |
| Mogamuizumab | Poteligeo | CCR4; Humanized IgG1 | Cutaneous T cell lymphoma |
| Olaratumab | Lartruvo | PDGRFα; Human IgG1 | Soft tissue sarcoma |
| Daratumumab | Darzalex | CD38; Human IgG1 | Multiple myeloma |
| Elotuzumab | Empliciti | SLAMF7; Humanized IgG1 | Multiple myeloma |
| Necitumumab | Portrazza | EGFR; Human IgG1 | Non-small cell lung cancer |
| Dinutuximab | Unituxin | GD2; Chimeric IgG1 | Neuroblastoma |
| Ramucirumab | Cyramza | VEGFR2; Human IgG1 | Gastric cancer |
| Obinutuzumab | Gazyva | CD20; Humanized IgG1; | Chronic lymphocytic leukemia |
| | | Glycoengineered | |
| Pertuzumab | Perjeta | HER2; Humanized IgG1 | Breast Cancer |
| Ofatumumab | Arzerra | CD20; Human IgG1 | Chronic lymphocytic leukemia |
| Panitumumab | Vectibix | EGFR; Human IgG2 | Colorectal cancer |
| Cetuximab | Erbitux | EGFR; Chimeric IgG1 | Colorectal cancer |
| Alemtuzumab | MabCampath, | CD52; Humanized IgG1 | Chronic myeloid leukemia#; |
| | Campath-1H; | | multiple sclerosis |
| | Lemtrada | | |
| Trastuzumab | Herceptin | HER2; Humanized IgG1 | Breast cancer |
| Rituximab | MabThera, | CD20; Chimeric IgG1 | Non-Hodgkin lymphoma |
| | Rituxan | | |
| Edrecolomab | Panorex | EpCAM; Murine IgG2a | Colorectal cancer |

### Antibodies

As used herein, the term “antibody” includes humanized, fully human or chimeric antibodies that contain the Fc region or bispecific antibodies designed to target NK cells, also known as natural killer cell engagers, where one arm of the bispecific antibody recognizes the tumor cell and the other recognizes and activating molecular on the NK cell such as CD16 (Fcgammar receptor III) or NKp46. In certain embodiments, the antibody is a human antibody or a humanized antibody. A “humanized” antibody contains only the three CDRs (complementarity determining regions) and sometimes a few carefully selected “framework” residues (the non-CDR portions of the variable regions) from each donor antibody variable region recombinantly linked onto the corresponding frameworks and constant regions of a human antibody sequence. A “fully humanized antibody” is created in a hybridoma from mice genetically engineered to have only human-derived antibody genes or by selection from a phage-display library of human-derived antibody genes.

As used herein, the term “monoclonal antibody” refers to an antibody obtained from a group of substantially homogeneous antibodies, that is, an antibody group wherein the antibodies constituting the group are homogeneous except for naturally occurring mutants that exist in a small amount. Monoclonal antibodies are highly specific and interact with a single antigenic site. Furthermore, each monoclonal antibody targets a single antigenic determinant (epitope) on an antigen, as compared to common polyclonal antibody preparations that typically contain various antibodies against diverse antigenic determinants. In addition to their specificity, monoclonal antibodies are advantageous in that they are produced from hybridoma cultures not contaminated with other immunoglobulins.

The adjective “monoclonal” indicates a characteristic of antibodies obtained from a substantially homogeneous group of antibodies, and does not specify antibodies produced by a particular method. For example, a monoclonal antibody to be used in the present invention can be produced by, for example, hybridoma methods. The monoclonal antibodies used in the present invention can be also isolated from a phage antibody library. The monoclonal antibodies of the present invention particularly comprise “chimeric” antibodies (immunoglobulins), wherein a part of a heavy (H) chain and/or light (L) chain is derived from a specific species or a specific antibody class or subclass, and the remaining portion of the chain is derived from another species, or another antibody class or subclass. Furthermore, mutant antibodies and antibody fragments thereof are also comprised in the present invention.

As used herein, the term “mutant antibody” refers to an antibody comprising a variant amino acid sequence in which one or more amino acid residues have been altered. For example, the variable region of an antibody can be modified to improve its biological properties, such as antigen binding. Such modifications can be achieved by site-directed mutagenesis, PCR-based mutagenesis, cassette mutagenesis, and the like. Such mutants comprise an amino acid sequence which is at least 70% identical to the amino acid sequence of a heavy or light chain variable region of the antibody, more preferably at least 75%, even more preferably at least 80%, still more preferably at least 85%, yet more preferably at least 90%, and most preferably at least 95% identical. As used herein, the term “sequence identity” is defined as the percentage of residues identical to those in the antibody's original amino acid sequence, determined after the sequences are aligned and gaps are appropriately introduced to maximize the sequence identity as necessary.

Specifically, the identity of one nucleotide sequence or amino acid sequence to another can be determined using the algorithm BLAST. Programs such as BLASTN and BLASTX were developed based on this algorithm. To analyze nucleotide sequences according to BLASTN based on BLAST, the parameters are set, for example, as score=100 and wordlength=12. On the other hand, parameters used for the analysis of amino acid sequences by BLASTX based on BLAST include, for example, score=50 and wordlength=3. Default parameters for each program are used when using the BLAST and Gapped BLAST programs. Specific techniques for such analyses are known in the art (see the website of the National Center for Biotechnology Information (NCBI), Basic Local Alignment Search Tool (BLAST)).

Monoclonal antibodies can be prepared by methods known to those skilled in the art.

In another embodiment, antibodies can be isolated from an antibody phage library. There are also reports that describe the production of high affinity (nM range) human antibodies based on chain shuffling, and combinatorial infection and in vivo recombination, which are methods for constructing large-scale phage libraries. These technologies can also be used to isolate monoclonal antibodies, instead of using conventional hybridoma technology for monoclonal antibody production.

Antibodies to be used in the present invention can be purified by a method appropriately selected from known methods, such as the protein A-Sepharose method, hydroxyapatite chromatography, salting-out method with sulfate, ion exchange chromatography, and affinity chromatography, or by the combined use of the same.

The present invention may use recombinant antibodies produced by gene engineering. The genes encoding the antibodies obtained by a method described above are isolated from the hybridomas. The genes are inserted into an appropriate vector, and then introduced into a host. The present invention provides the nucleic acids encoding the antibodies of the present invention, and vectors comprising these nucleic acids. Specifically, using a reverse transcriptase, cDNAs encoding the variable regions (V regions) of the antibodies are synthesized from the mRNAs of hybridomas. After obtaining the DNAs encoding the variable regions of antibodies of interest, they are ligated with DNAs encoding desired constant regions (C regions) of the antibodies, and the resulting DNA constructs are inserted into expression vectors. Alternatively, the DNAs encoding the variable regions of the antibodies may be inserted into expression vectors comprising the DNAs of the antibody C regions. These are inserted into expression vectors so that the genes are expressed under the regulation of an expression regulatory region, for example, an enhancer and promoter. Then, host cells are transformed with the expression vectors to express the antibodies. The present invention provides cells expressing antibodies of the present invention. The cells expressing antibodies of the present invention include cells and hybridomas transformed with a gene of such an antibody.

In certain embodiments, an amino acid residue is mutated into one that allows the properties of the amino acid side-chain to be conserved. Examples of the properties of amino acid side chains comprise: hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K, H); and aromatic-containing side-chains (H, F, Y, W). The letters within parenthesis indicate the one-letter amino acid codes. Amino acid substitutions within each group are called conservative substitutions. It is well known that a polypeptide comprising a modified amino acid sequence in which one or more amino acid residues is deleted, added, and/or substituted can retain the original biological activity. The number of mutated amino acids is not limited, but in general, the number falls within 40% of amino acids of each CDR, and preferably within 35%, and still more preferably within 30% (e.g., within 25%). The identity of amino acid sequences can be determined as described herein.

In the present invention, recombinant antibodies artificially modified to reduce heterologous antigenicity against humans can be used. Examples include chimeric antibodies and humanized antibodies. These modified antibodies can be produced using known methods. A chimeric antibody includes an antibody comprising variable and constant regions of species that are different to each other, for example, an antibody comprising the antibody heavy chain and light chain variable regions of a nonhuman mammal such as a mouse, and the antibody heavy chain and light chain constant regions of a human. Such an antibody can be obtained by (1) ligating a DNA encoding a variable region of a mouse antibody to a DNA encoding a constant region of a human antibody; (2) incorporating this into an expression vector; and (3) introducing the vector into a host for production of the antibody.

A humanized antibody, which is also called a reshaped human antibody, is obtained by substituting an H or L chain complementarity determining region (CDR) of an antibody of a nonhuman mammal such as a mouse, with the CDR of a human antibody. Conventional genetic recombination techniques for the preparation of such antibodies are known. Specifically, a DNA sequence designed to ligate a CDR of a mouse antibody with the framework regions (FRs) of a human antibody is synthesized by PCR, using several oligonucleotides constructed to comprise overlapping portions at their ends. A humanized antibody can be obtained by (1) ligating the resulting DNA to a DNA that encodes a human antibody constant region; (2) incorporating this into an expression vector; and (3) transfecting the vector into a host to produce the antibody (see, European Patent Application No. EP 239,400, and International Patent Application No. WO 96/02576). Human antibody FRs that are ligated via the CDR are selected where the CDR forms a favorable antigen-binding site. The humanized antibody may comprise additional amino acid residue(s) that are not included in the CDRs introduced into the recipient antibody, nor in the framework sequences. Such amino acid residues are usually introduced to more accurately optimize the antibody's ability to recognize and bind to an antigen. For example, as necessary, amino acids in the framework region of an antibody variable region may be substituted such that the CDR of a reshaped human antibody forms an appropriate antigen-binding site.

The isotypes of the antibodies of the present invention are not limited. The isotypes include, for example, IgG (IgG1, IgG2, IgG3, and IgG4), IgM, IgA (IgA1 and IgA2), IgD, and IgE. The antibodies of the present invention may also be bispecific antibodies (also known as NK cell engagers) that are specific for both the cancer cell and molecules on NK cells such as CD16 and NKp46 intended to mediate NK cell ADCC.

The term “diabody (db)” refers to a bivalent antibody fragment constructed by gene fusion. In general, a diabody is a dimer of two polypeptide chains. In the each of the polypeptide chains, a light chain variable region (V_{L}) and a heavy chain variable region (V_{H}) in an identical chain are connected via a short linker, for example, a linker of about five residues, so that they cannot bind together. Because the linker between the two is too short, the V_{L} and V_{H} in the same polypeptide chain cannot form a single chain V region fragment, but instead form a dimer. Thus, a diabody has two antigen-binding domains. When the V_{L} and V_{H} regions against the two types of antigens (a and b) are combined to form V_{La}-V_{Hb} and V_{Lb}-V_{Ha} via a linker of about five residues, and then co-expressed, they are secreted as bispecific Dbs. The antibodies of the present invention may be such Dbs.

A single-chain antibody (also referred to as “scFv”) can be prepared by linking a heavy chain V region and a light chain V region of an antibody. Methods for preparing single-chain antibodies are known in the art. In such scFvs, the heavy chain V region and the light chain V region are linked together via a linker, preferably, a polypeptide linker). The heavy chain V region and the light chain V region in a scFv may be derived from the same antibody, or from different antibodies. The peptide linker used to ligate the V regions may be any single-chain peptide consisting of 12 to 19 residues. A DNA encoding a scFv can be amplified by PCR using, as a template, either the entire DNA, or a partial DNA encoding a desired amino acid sequence, selected from a DNA encoding the heavy chain or the V region of the heavy chain of the above antibody, and a DNA encoding the light chain or the V region of the light chain of the above antibody; and using a primer pair that defines the two ends. Further amplification can be subsequently conducted using a combination of the DNA encoding the peptide linker portion, and the primer pair that defines both ends of the DNA to be ligated to the heavy and light chain respectively. After constructing DNAs encoding scFvs, conventional methods can be used to obtain expression vectors comprising these DNAs, and hosts transformed by these expression vectors. Furthermore, scFvs can be obtained according to conventional methods using the resulting hosts. These antibody fragments can be produced in hosts by obtaining genes that encode the antibody fragments and expressing these as outlined above. Antibodies bound to various types of molecules, such as polyethylene glycols (PEGs), may be used as modified antibodies. Methods for modifying antibodies are already established in the art. The term “antibody” in the present invention also encompasses the above-described antibodies.

The antibodies obtained can be purified to homogeneity. The antibodies can be isolated and purified by a method routinely used to isolate and purify proteins. The antibodies can be isolated and purified by the combined use of one or more methods appropriately selected from column chromatography, filtration, ultrafiltration, salting out, dialysis, preparative polyacrylamide gel electrophoresis, and isoelectro-focusing, for example. Such methods are not limited to those listed above. Chromatographic methods include affinity chromatography, ion exchange chromatography, hydrophobic chromatography, gel filtration, reverse-phase chromatography, and adsorption chromatography. These chromatographic methods can be practiced using liquid phase chromatography, such as HPLC and FPLC. Columns to be used in affinity chromatography include protein A columns and protein G columns. For example, protein A columns include Hyper D, POROS, and Sepharose F. F. (Pharmacia). Antibodies can also be purified by utilizing antigen binding, using carriers on which antigens have been immobilized.

The antibodies of the present invention can be formulated according to standard methods (see, for example, Remington's Pharmaceutical Science, latest edition, Mark Publishing Company, Easton, U.S.A), and may comprise pharmaceutically acceptable carriers and/or additives. The present invention relates to compositions (including reagents and pharmaceuticals) comprising the antibodies of the invention, and pharmaceutically acceptable carriers and/or additives. Exemplary carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed in the present invention are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and immunoglobulin, and amino acids such as glycine, glutamine, asparagine, arginine, and lysine. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50).

If necessary, antibodies of the present invention may be encapsulated in microcapsules (microcapsules made of hydroxycellulose, gelatin, polymethylmethacrylate, and the like), and made into components of colloidal drug delivery systems (liposomes, albumin microspheres, microemulsions, nano-particles, and nano-capsules). Moreover, methods for making sustained-release drugs are known, and these can be applied for the antibodies of the present invention.

### Pharmaceutical Compositions

Pharmaceutical compositions or formulations of the present invention include combinations of an anti-cancer antibody, an T cell activating agent, and one or more pharmaceutically acceptable carrier, glidant, diluent, or excipient.

Pharmaceutical compositions encompass both the bulk composition and individual dosage units comprised of more than one (e.g., two) pharmaceutically active agents including an anti-cancer antibody, an T cell activating agent, along with any pharmaceutically inactive excipients, diluents, carriers, or glidants. The bulk composition and each individual dosage unit can contain fixed amounts of the aforesaid pharmaceutically active agents. The bulk composition is material that has not yet been formed into individual dosage units. An illustrative dosage unit is an oral dosage unit such as tablets, pills, capsules, and the like. Similarly, the herein-described method of treating a patient by administering a pharmaceutical composition of the present invention is also intended to encompass the administration of the bulk composition and individual dosage units.

### Formulations and Methods of Administration

In certain embodiments, an effective amount of the therapeutic composition is administered to the subject. “Effective amount” or “therapeutically effective amount” are used interchangeably herein, and refer to an amount of a compound, formulation, material, or composition, as described herein effective to achieve a particular biological result. Such results may include, but are not limited to the inhibition of cancer as determined by any means suitable in the art.

Anti-cancer antibodies and T cell activating agents are formulated in accordance with standard pharmaceutical practice for use in a therapeutic combination for therapeutic treatment (including prophylactic treatment) of hyperproliferative disorders in mammals including humans. The invention provides a pharmaceutical composition comprising a T cell activating agent in association with one or more pharmaceutically acceptable carrier, glidant, diluent, or excipient.

Suitable carriers, diluents and excipients are well known to those skilled in the art and include materials such as carbohydrates, waxes, water soluble and/or swellable polymers, hydrophilic or hydrophobic materials, gelatin, oils, solvents, water and the like. The particular carrier, diluent or excipient used will depend upon the means and purpose for which the compound of the present invention is being applied. Solvents are generally selected based on solvents recognized by persons skilled in the art as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound of the present invention or pharmaceutical composition thereof) or aid in the manufacturing of the pharmaceutical product (i.e., medicament).

The formulations may be prepared using conventional dissolution and mixing procedures. For example, the bulk drug substance (i.e., agent of the present invention or stabilized form of the compound (e.g., complex with a cyclodextrin derivative or other known complexation agent) is dissolved in a suitable solvent in the presence of one or more of the excipients described above. The agent is typically formulated into pharmaceutical dosage forms to provide an easily controllable dosage of the drug and to enable patient compliance with the prescribed regimen.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Pharmaceutical formulations of the compounds of the present invention may be prepared for various routes and types of administration. For example, an agent having the desired degree of purity may optionally be mixed with pharmaceutically acceptable diluents, carriers, excipients or stabilizers (Remington's Pharmaceutical Sciences (1995) 18th edition, Mack Publ. Co., Easton, PA), in the form of a lyophilized formulation, milled powder, or an aqueous solution. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8.

The pharmaceutical formulation is preferably sterile. In particular, formulations to be used for in vivo administration must be sterile. Such sterilization is readily accomplished by filtration through sterile filtration membranes.

The pharmaceutical formulation ordinarily can be stored as a solid composition, a lyophilized formulation or as an aqueous solution.

The pharmaceutical formulations will be dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the agent to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

As a general proposition, the initial pharmaceutically effective amount of the agent administered orally or parenterally per dose will be in the range of about 0.01-1000 mg/kg, namely about 0.1 to 20 mg/kg of patient body weight per day, with the typical initial range of compound used being 0.3 to 15 mg/kg/day. The dose of the anti-cancer antibody and the dose of the T cell activating agent to be administered may range for each from about 2 micrograms to about 1000 mg per unit dosage form. The dose of the anti-cancer antibody and the dose of the T cell activating agent may administered in a ratio of about 1:50 to about 50:1 by weight, or in a ratio of about 1:10 to about 10:1 by weight.

Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 18th edition, (1995) Mack Publ. Co., Easton, PA.

Sustained-release preparations of the T cell activating agent may be prepared. Suitable examples of sustained-release preparations include semipermeable matrices of solid hydrophobic polymers containing the T cell activating agent, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D (−) 3-hydroxybutyric acid.

The pharmaceutical formulations include those suitable for the administration routes detailed herein. The formulations may conveniently be presented in unit dosage form and may be prepared by any of the methods well known in the art of pharmacy. Techniques and formulations generally are found in Remington's Pharmaceutical Sciences 18^{th} Ed. (1995) Mack Publishing Co., Easton, PA. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations of the anti-cancer antibody and/or T cell activating agent suitable for oral administration may be prepared as discrete units such as pills, hard or soft e.g., gelatin capsules, cachets, troches, lozenges, aqueous or oil suspensions, dispersible powders or granules, emulsions, syrups or elixirs each containing a predetermined amount of the anti-cancer antibody and/or T cell activating agent. The amount of anti-cancer antibody and the amount of T cell activating agent may be formulated in a pill, capsule, solution or suspension as a combined formulation. Alternatively, the anti-cancer antibody and the T cell activating agent may be formulated separately in a pill, capsule, solution or suspension for administration by alternation.

Formulations may be prepared according to any method known to the art for the manufacture of pharmaceutical compositions and such compositions may contain one or more agents including sweetening agents, flavoring agents, coloring agents and preserving agents, in order to provide a palatable preparation. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablet excipients of a pharmaceutical formulation may include: Filler (or diluent) to increase the bulk volume of the powdered drug making up the tablet; Disintegrants to encourage the tablet to break down into small fragments, ideally individual drug particles, when it is ingested and promote the rapid dissolution and absorption of drug; Binder to ensure that granules and tablets can be formed with the required mechanical strength and hold a tablet together after it has been compressed, preventing it from breaking down into its component powders during packaging, shipping and routine handling; Glidant to improve the flowability of the powder making up the tablet during production; Lubricant to ensure that the tableting powder does not adhere to the equipment used to press the tablet during manufacture. They improve the flow of the powder mixes through the presses and minimize friction and breakage as the finished tablets are ejected from the equipment; Antiadherent with function similar to that of the glidant, reducing adhesion between the powder making up the tablet and the machine that is used to punch out the shape of the tablet during manufacture; Flavor incorporated into tablets to give them a more pleasant taste or to mask an unpleasant one, and Colorant to aid identification and patient compliance.

Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of the emulsions of this invention may be constituted from known ingredients in a known manner, including a mixture of at least one emulsifier with a fat or an oil, or with both a fat and an oil. In certain embodiments, a hydrophilic emulsifier is included together with a lipophilic emulsifier which acts as a stabilizer. Together, the emulsifier(s) with or without stabilizer(s) make up an emulsifying wax, and the wax together with the oil and fat comprise an emulsifying ointment base which forms the oily dispersed phase of cream formulations. Emulsifiers and emulsion stabilizers suitable for use in the formulation include Tween® 60, Span® 80, cetostearyl alcohol, benzyl alcohol, myristyl alcohol, glyceryl mono-stearate and sodium lauryl sulfate.

Aqueous suspensions of the pharmaceutical formulations contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxybenzoate, one or more coloring agents, one or more flavoring agents and one or more sweetening agents, such as sucrose or saccharin.

Pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may be a solution or a suspension in a non-toxic parenterally acceptable diluent or solvent, such as a solution in 1,3-butanediol or prepared from a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis disorders as described below.

Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The invention further provides veterinary compositions comprising at least one active ingredient as above defined together with a veterinary carrier therefor. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Combination Therapy

The T cell activating agent may be employed in combination with an anti-cancer antibody for the treatment of a hyperproliferative disease or disorder, including tumors, cancers, and neoplastic tissue, along with pre-malignant and non-neoplastic or non-malignant hyperproliferative disorders. In certain embodiments, a T cell activating agent is combined in a dosing regimen as combination therapy, with an anti-cancer antibody. The T cell activating agent of the dosing regimen preferably has complementary activities to the anti-cancer antibody, and such that they do not adversely affect each other. Such compounds may be administered in amounts that are effective for the purpose intended. In one embodiment, the therapeutic combination is administered by a dosing regimen wherein the therapeutically effective amount of an anti-cancer antibody, or a pharmaceutically acceptable salt thereof is administered in a range from twice daily to once every four weeks (q3 wk), and the therapeutically effective amount of the T cell activating agent is administered in a range from twice daily to once every four weeks.

The combination therapy may be administered as a simultaneous or sequential regimen. When administered sequentially, the combination may be administered in two or more administrations. The combined administration includes coadministration, using separate formulation, and consecutive administration in either order, wherein preferably there is a time period while both (or all) active agents simultaneously exert their biological activities.

In one specific aspect of the invention, the anti-cancer antibody can be administered for a time period of about 1 to about 10 days after administration of the one or more agents begins. In another specific aspect of the invention, the anti-cancer antibody can be administered for a time period of about 1 to 10 days before administration of the combination begins. In another specific aspect of the invention, administration of the anti-cancer antibody and administration of the T cell activating agent begin on the same day.

Suitable dosages for any of the above co-administered agents are those presently used and may be lowered due to the combined action (synergy) of the newly identified agent and other T cell activating agents or treatments, such as to increase the therapeutic index or mitigate toxicity or other side-effects or consequences.

In a particular embodiment of anti-cancer therapy, an anti-cancer antibody, may be combined with a T cell activating agent, as well as combined with surgical therapy and radiotherapy. The amounts of the anti-cancer antibody and the other pharmaceutically active chemotherapeutic agent(s) and the relative timings of administration will be selected in order to achieve the desired combined therapeutic effect.

The combination may be administered along with other anti-cancer agents including chemotherapy, radiation therapy, targeted therapy or cancer immunotherapy agents.

### Administration of Pharmaceutical Compositions

The antibodies and agents may be administered by any route appropriate to the condition to be treated. Suitable routes include oral, parenteral (including subcutaneous, intramuscular, intravenous, intraarterial, inhalation, intradermal, intrathecal, epidural, and infusion techniques), transdermal, rectal, nasal, topical (including buccal and sublingual), vaginal, intraperitoneal, intrapulmonary and intranasal. Topical administration can also involve the use of transdermal administration such as transdermal patches or iontophoresis devices.

Formulation of drugs is discussed in Remington's Pharmaceutical Sciences, 18^{th} Ed., (1995) Mack Publishing Co., Easton, PA. Other examples of drug formulations can be found in Liberman, H. A. and Lachman, L., Eds., Pharmaceutical Dosage Forms, Marcel Decker, Vol 3, 2^{nd} Ed., New York, NY. For local immunosuppressive treatment, the antibodies and agents may be administered by intralesional administration, including perfusing or otherwise contacting the graft with the inhibitor before transplantation. It will be appreciated that the preferred route may vary with for example the condition of the recipient. Where the antibodies or agents are administered orally, it may be formulated as a pill, capsule, tablet, etc. with a pharmaceutically acceptable carrier, glidant, or excipient. Where the antibodies or agents are administered parenterally, they may be formulated with a pharmaceutically acceptable parenteral vehicle or diluent, and in a unit dosage injectable form, as detailed below.

A dose may be administered once a day (QD), twice per day (BID), or more frequently, depending on the pharmacokinetic (PK) and pharmacodynamic (PD) properties, including absorption, distribution, metabolism, and excretion of the particular compound. In addition, toxicity factors may influence the dosage and administration dosing regimen. When administered orally, the pill, capsule, or tablet may be ingested twice daily, daily or less frequently such as weekly or once every two or three weeks for a specified period of time. The regimen may be repeated for a number of cycles of therapy.

### Methods of Treatment

Therapeutic combinations of: (1) an anti-cancer antibody and (2) a T cell activating agent are useful for treating diseases, conditions and/or disorders including, but not limited to, hyperproliferative disorders, including cancers. Cancers which can be treated according to the methods of this invention include, but are not limited to, B cell lymphoma, T cell lymphoma, myeloma, non-small cell lung cancer, small cell lung cancer, breast cancer, head and neck cancer, neuroblastoma, soft tissue sarcoma, gastric cancer, colorectal cancer, chronic lymphocytic leukemia, acute lymphoblastic leukemia, chronic myeloid leukemia, acute myeloid leukemia, pancreatic cancer, and prostate cancer.

### Articles of Manufacture

In another embodiment of the invention, an article of manufacture, or “kit”, containing a T cell activating agent useful for the treatment of the diseases and disorders described above is provided. In one embodiment, the kit comprises a container and a compound of T cell activating agent. In one embodiment, the kit also contains an anti-cancer antibody.

The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container may hold a T cell activating agent, or a formulation thereof which is effective for treating the condition and may have a sterile access port (for example, the container may be an intravenous solution bag or a vial having a stopper pierceable by a hypodermic injection needle). At least one active agent in the composition is a T cell activating agent. The label or package insert indicates that the composition is used for treating the condition of choice, such as cancer. In one embodiment, the label or package inserts indicates that the composition comprising a T cell activating agent can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of a first pharmaceutical composition (e.g., the T cell activating agent), and, if present, the second pharmaceutical formulation (e.g., an anti-cancer antibody). For example, if the kit comprises a first composition comprising T cell activating agent and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of a T cell activating agent, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with a T cell activating agent contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Where the kit comprises a composition of a T cell activating agent and a second therapeutic agent, i.e. the chemotherapeutic agent, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

The invention will now be illustrated by the following non-limiting Examples.

### Example 1

### T Cells, Particularly Activated CD4⁺ Cells, Maintain Anti-CD20-Mediated NK Cell Viability and Antibody Dependent Cellular Cytotoxicity

### Background

Therapeutic anti-tumor monoclonal antibody (mAb) became a standard and important component of cancer therapy starting with FDA approval of rituximab (RTX), a mouse-human chimeric anti-CD20 mAb, in 1997. However, some B cell malignancies fail to respond or recur following anti-CD20-based therapy. Understanding the mechanism of action by which RTX mediates its anti-tumor efficacy is critical for improving its efficacy. Multiple mechanisms have been proposed for RTX, including antibody-dependent cellular cytotoxicity (ADCC), complement dependent cytotoxicity, direct apoptosis and phagocytosis. The clinical observation that follicular lymphoma (FL) patients with high affinity CD16 V158F polymorphism respond better to RTX than those with low affinity provides one compelling piece of evidence that NK cell-mediated ADCC is central to mediating the anti-tumor activity of RTX in humans.

Human NK cells are typically defined as CD3-CD56⁺ lymphocytes and are divided into two major subsets: CD56^{dim} and CD56^{bright}. CD56^{dim} NK cells make up the majority (˜90%) of circulating NK cells and express high levels of CD16 (FcγTRIIIa). They are considered to be the main effectors for ADCC. A smaller subset of NK cells is CD56^{bright} and generally express lower levels of CD16. They regulate the immune function mainly via cytokine production. While there is debate over the origin and relationship between CD56^{dim} and CD56^{bright} NK cells, the widely accepted paradigm is that CD56^{dim} NK are more mature and evolve from CD56^{bright} NK. During this differentiation process from CD56^{bright} to CD56^{dim}, NK cells decrease the expression of c-kit, CD127 and CD62L while increasing the expression of CD57, KIRs and CD16. Functionally, CD56^{dim} NK cells gain more cytotoxicity and gradually lose proliferative potential.

The short-term effects of RTX on NK cells using 4 or 20-hour assays have been extensively studied. However, the median half-life of RTX in non-Hodgkin lymphoma patients is 76 hours and 206 hours respectively after the first and fourth infusion. The long-term effect of RTX on NK cells remains an important but understudied question. In addition, there is significant interest in understanding the cross talk between NK cells and T cells based on the durable protection from the tumor observed in some FL patients receiving RTX treatment that suggests a vaccinal effect. Mechanistically, tumor bound anti-CD20 mAb activates NK cells and promotes the maturation of dendritic cells (DC). Mature DC and activated NK cells synergistically prime naïve T cells to induce a Th1 response and form T cell memory. While the effect of NK cell activation on T cells has been explored, the opposite, namely the effect of T cells on NK cells following RTX therapy, has received little attention.

Here, the long-term effect of RTX, and other anti-cancer monoclonal antibodies, on NK cell responses was evaluated, as well as the effect of T cells on that response.

### Methods

Samples and reagents. Peripheral blood mononuclear cells (PBMC) of healthy donors were isolated from leukocyte reduction system cones (DeGowin Blood Center, the University of Iowa) using Ficoll-Paque gradient centrifugation. Following informed consent, Holden Comprehensive Cancer Center patients receiving weekly single agent RTX treatment provided peripheral blood collected before the 1^{st}, 2^{nd}, and 4^{th} RTX infusion for analysis. The use of human samples was approved by the Institutional Review Board. Raji B cell lymphoma cells were obtained from ATCC. SQ20B cells were kindly provided by Andrean Simons-Burnett in the Department of Pathology, the University of Iowa. RTX, obinutuzumab (OBZ), cetuximab (CTX) and trastuzumab (TRA) were from University of Iowa Hospitals & Clinics. 1DT3D was developed as previously reported. CellTrace CFSE, CellTracker Red CMTPX dye, Dynabeads Human T-Activator anti-CD3/28 and CountBright Absolute counting beads were from Thermo Fisher.

Cell coculture. For most analyses, 1 million PBMC were co-cultured with 0.2 million Raji cells after addition of RTX, OBZ or TRA at a concentration of 1 ug/ml in a total volume of 200 ul in round bottom 96-well plates (Corning). For the Transwell assay, the 96-well Transwell system (lum pore size, Corning) was used to separate T cells from T cell-depleted PBMCs. RMPI was supplemented with 10% FBS, 100 U/ug/ml Penicillin/Streptomycin, 2 mM L-Glutamine and 50 mM β-mercaptoethanol to make complete cell culture medium. α-IL2 (clone: 5334, R&D), α-IFNγ(clone: B27, BioLegend), α-CD54 (clone: HCD54, BioLegend), α-FGFR1(clone: 133111, R&D) mAb, α-CD16 polyclonal Ab (R&D) or recombinant human IL2 (PeproTech) was added to the coculture in specific experimental settings. Complete medium including Abs or IL2 was refreshed every 2 days. Experiments were independently performed at least three times.

Cell purification and depletion. Cell isolation kits (Miltenyi Biotec) were used to isolate NK cells (#130-092-657), B cells (#130-101-638), CD3⁺ T cells (#130-096-535), CD4⁺ T cells (#130-096-533), and CD8⁺ T cells (#130-096-495) by negative selection. Microbeads (Miltenyi Biotec) were used to deplete CD14⁺ monocytes (#130-050-201), CD19⁺ B cells (#130-050-301), CD3⁺ (#130-050-101), CD4⁺ (#130-045-101) or CD8⁺ T (#130-045-201) cells by LD columns. To separate CD56^{dim} from CD56^{bright} NK cells, NK cells were isolated from PBMC by negative selection and sorted using a Becton Dickinson Aria II flow cytometer.

Flow cytometry. Staining antibodies (BioLegend) included anti-human CD3 (HIT3a), CD56 (HCD56), CD14 (HCD14), CD19 (HIB19), CD16 (3G8), CD57 (HNK-1), KIR2DL1/S1/S3/S5 (HP-MA4), CD69 (FN50), CD25 (M-A251), CD54 (HCD54). Cells were first washed with PBS and stained with Zombie Aqua Fixable viability dye (BioLegend) to distinguish dead cells, followed by incubation with staining antibodies for 15 minutes at 4° C. Samples were fixed in 2% formaldehyde and read within 24 hours in the flow cytometry facility in the University of Iowa. Data were analyzed using FlowJo v10.7 (FlowJo LLC).

Proliferation assay. Cells were washed with PBS and stained with 5 uM CFSE per manufacturer recommendations. Proliferation was quantified by determining the percent of cells with CFSE dilution. The absolute cell count was calculated using flow counting beads.

mRNA sequencing. NK cells were isolated from the Raji-RTX-Effector coculture by negative selection and flow cytometric sorting. RNA was extracted from isolated NK cells using RNeasy Mini Kit (QIAGEN). Transcription profiling using RNA-Seq was performed by the University of Iowa Genomics Division using manufacturer recommended protocols. Briefly, 500 ng of DNase I-treated total RNA was used to enrich for poly-A containing transcripts using oligo(dT) primers bound to beads. The enriched RNA pool was then fragmented, converted to cDNA and ligated to sequencing adaptors containing indexes using the Illumina TruSeq stranded mRNA sample preparation kit (Illumina). The molar concentrations of the indexed libraries were measured using the 2100 Agilent Bioanalyzer and combined equally into pools for sequencing. The concentration of the pools was measured using the Illumina Library Quantification Kit (KAPA Biosystems) and sequenced on the Illumina NovaSeq 6000 genome sequencer using 100 bp paired end SBS chemistry.

Bioinformatics analysis. Sequencing reads were processed with ‘bcbio-nextgen’ (v1.2.2) for QC, alignment, and read quantitation. The bcbio-nextgen pipeline was used to run ‘MultiQC’ and ‘qualimap’ (2.2.2), computational tools that examined SAM/BAM alignment files and detected common QC problems. Read were aligned against the GRCh37 reference genome using ‘hisat2’ aligner (2.2.0) and concurrently quantified reads to the transcriptome using the alignment-free tool ‘salmon’ (1.1.0) aligner. Following alignment, transcript expression estimates (TPM) were summarized from ‘salmon’ output to the gene level using ‘tximport’ (1.12.3) in R. Genes with fewer than five gene-level counts across all samples were excluded from downstream analysis. Differential gene expression analysis was conducted using DESeq2 (1.24.0). An FDR of 1% was set as a cutoff for differential expression genes (DEGs). Principal component analysis (PCA) plots were created with ‘pcaExplorer’. The DE gene data were analyzed using Advaita Bio's iPathwayGuide. This software analysis tool implements an ‘Impact Analysis’ approach that takes into consideration the direction and type of all signals on a pathway, the position, role and type of every gene as described.

Statistics. Paired or unpaired Student's t-test was used to compare two independent groups. One-way or two-way ANOVA was used for multiple comparisons between different groups. Data was presented as mean±SEM. All the analysis was performed in GraphPad Prism8. P<0.05 was considered to be statistically significant. * indicates p<0.05, **p<0.01, p<0.001, **** p<0.0001, ns: not significant.

### Results

### Long-Term Exposure to RTX Impacts on NK Cell Function and Phenotype

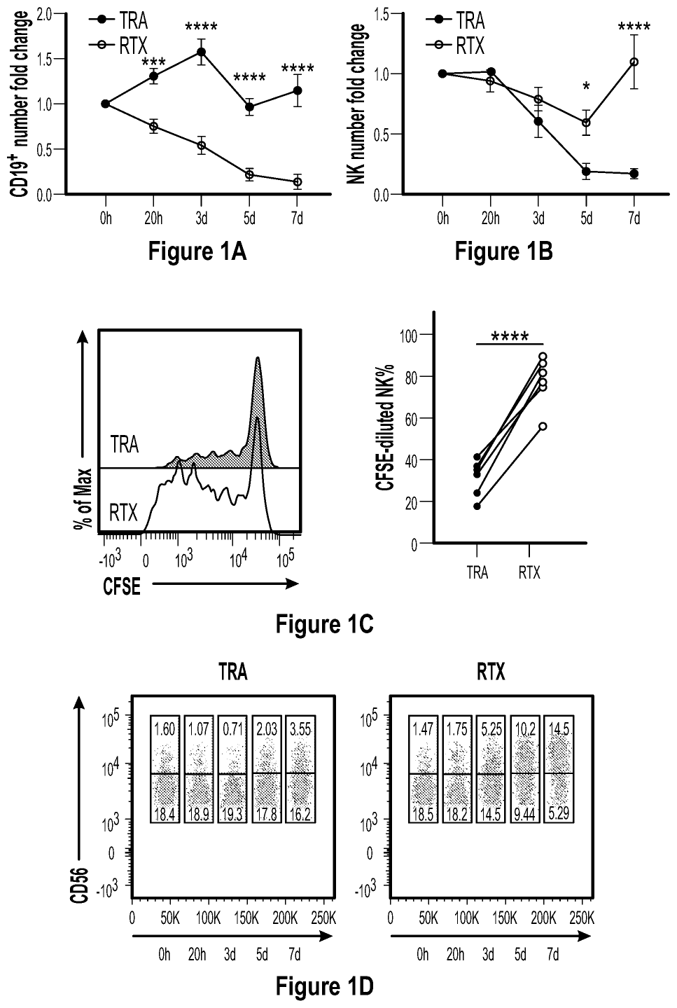

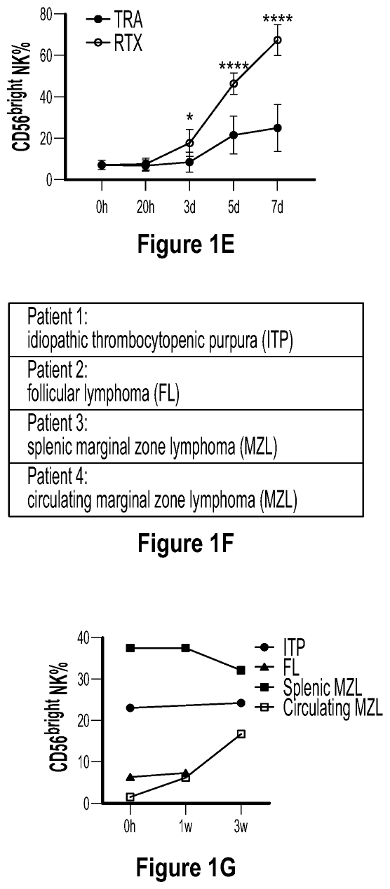

Initial studies evaluated the impact of RTX on ADCC as well as the number and phenotype of NK cells in longer term cultures of PBMCs and Raji lymphoma cells. The elimination of CD19⁺ target cells by RTX over time suggested ongoing ADCC (FIG. 1A). NK cells in these cultures persisted and proliferated as demonstrated by the maintenance in NK cell number (FIG. 1B) and CFSE dilution (FIG. 1C). After 5-7 days, NK cells within PBMCs cultured with Raji cells and RTX shifted from CD56^{dim} to CD56^{bright} (FIG. 1D, E). This change was not seen when TRA was added instead of RTX. OBZ, an anti-CD20 mAb recognizing a different-oriented epitope from RTX, showed changes consistent with those seen with RTX (FIGS. 8A, 8B). Change in NK cell phenotype in vivo was determined using peripheral blood samples from patients receiving weekly single agent RTX (FIG. 1F). The fraction of NK cells with the CD56^{bright} phenotype increased following RTX in the patient with circulating malignant cells but not in the patients without circulating malignant cells (FIG. 1G, FIG. 8C).

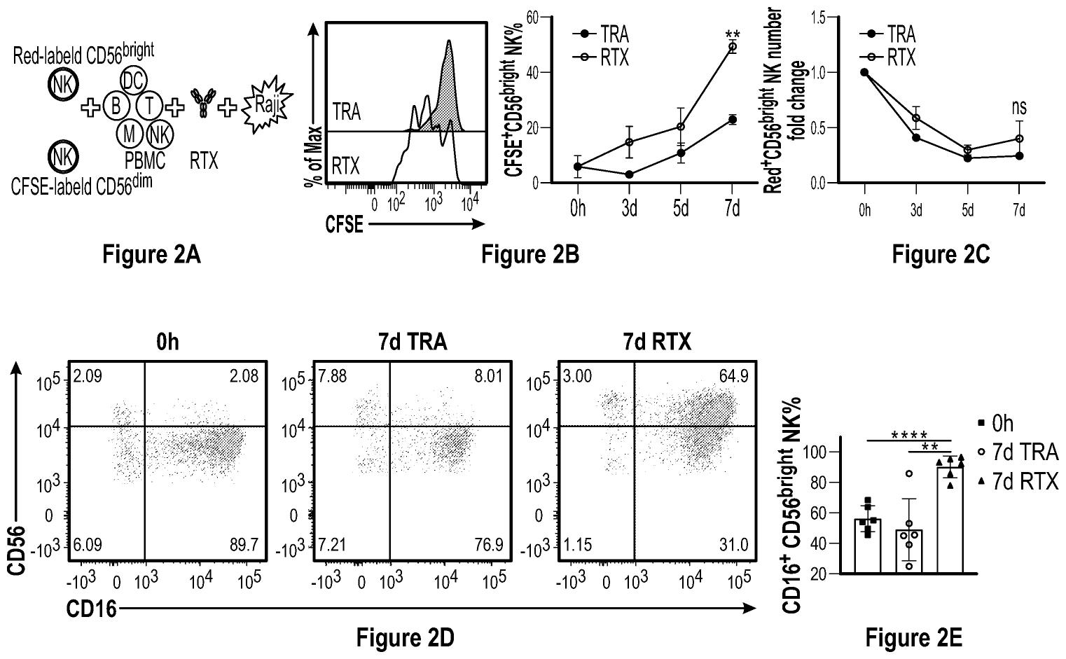

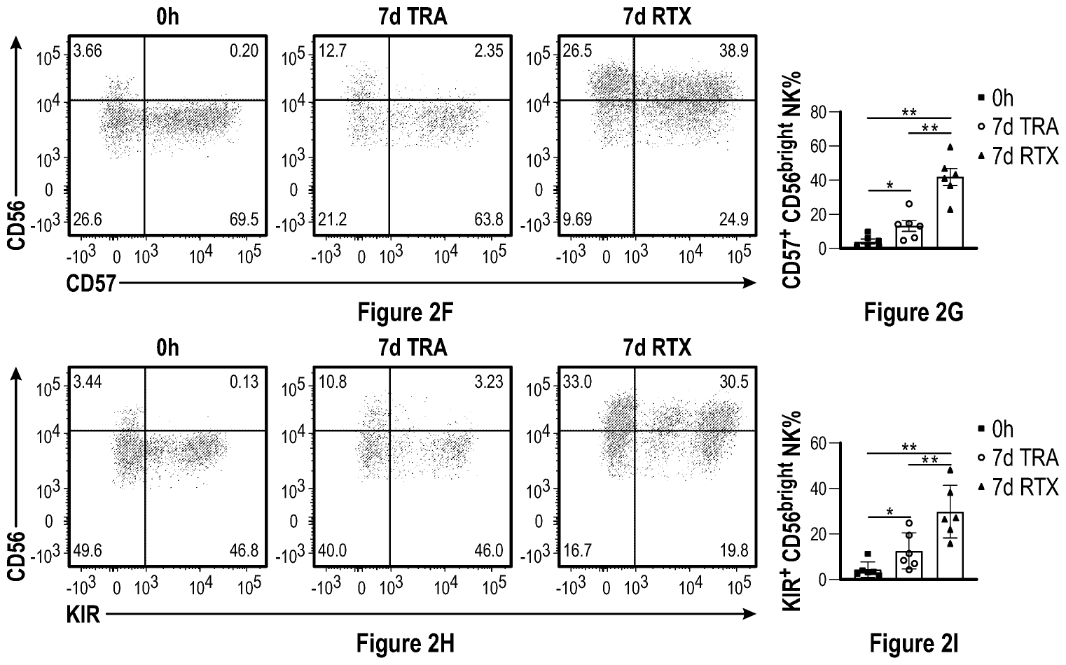

Further studies were done to assess whether the shift in CD56^{dim} and CD56^{bright}NK cells derived from the differential expansion of the two subsets, or a shift of CD56^{dim} to CD56^{bright} cells. Sorted CD56^{dim} and CD56^{bright} NK cells were labeled with CFSE and CellTracker Red respectively. Labeled CD56^{dim} and CD56^{bright} NK cells were added back to autologous PBMC and cultured with Raji cells and RTX (FIG. 2A). After 7 days, CFSE-labeled CD56^{dim} NK cells proliferated and displayed a CD56^{bright} immunophenotype (FIG. 2B) while CellTracker Red-labeled CD56^{bright} cells did not expand (FIG. 2C). These studies demonstrate CD56^{dim} NK cells within PBMCs proliferate and express more CD56 after longer term culture with Raji cells and RTX.

CD56^{bright} NK cells are classically considered to be immature and to differentiate into the mature CD56^{dim} subsets. Additional maturation markers were assessed to better understand the differentiation status of the CD56^{bright} NK cells that emerge following longer-term culture with RTX. Resting CD56^{bright} NK cells express low levels of CD16 while resting CD56^{dim} NK cells express higher levels of CD16. The expression of CD16 by CD56^{dim} is known to be downregulated on NK cells in response to short-term RTX activation. Indeed, downmodulation of CD16 was seen after 20 hours of co-culture with RTX and Raji. However, expression of CD16 on NK cells recovered after 7 days with the majority of RTX-activated NK cells expressing both CD56^{bright} and CD16 (FIG. 2D, E). These cells also expressed CD57 and KIR (FIG. 2F-I) which, in the resting state, are expressed largely by CD56^{dim} NK cells. Together, this phenotypic data suggests that RTX-activated CD56^{dim} NK cells upregulate the expression of CD56, re-express high levels of CD16, and display other markers of mature NK cells.

### T Cells are Required for Maintaining RTX-Mediated NK Cell Responses

NK cells were isolated from PBMC and cocultured with RTX and Raji cells for 7 days. In contrast to what was observed with NK cells in unfractionated PBMCs, RTX failed to induce CD56^{dim} to CD56^{bright} transition, CFSE dilution or CD16 re-expression by isolated NK cells (FIGS. 9A-9D). The number of NK cells remaining in the RTX group was higher than that in the TRA group, but this difference was considerably less than was seen with unfractionated PBMCs (RTX to TRA NK ratio—3.05 versus 9.42, FIG. 9E). The elimination of Raji cells by RTX was limited when isolated NK cells were used as effector cells (FIG. 9F). This suggested a cell population in PBMC was maintaining NK cell growth, viability, cytotoxicity, and phenotypic change.

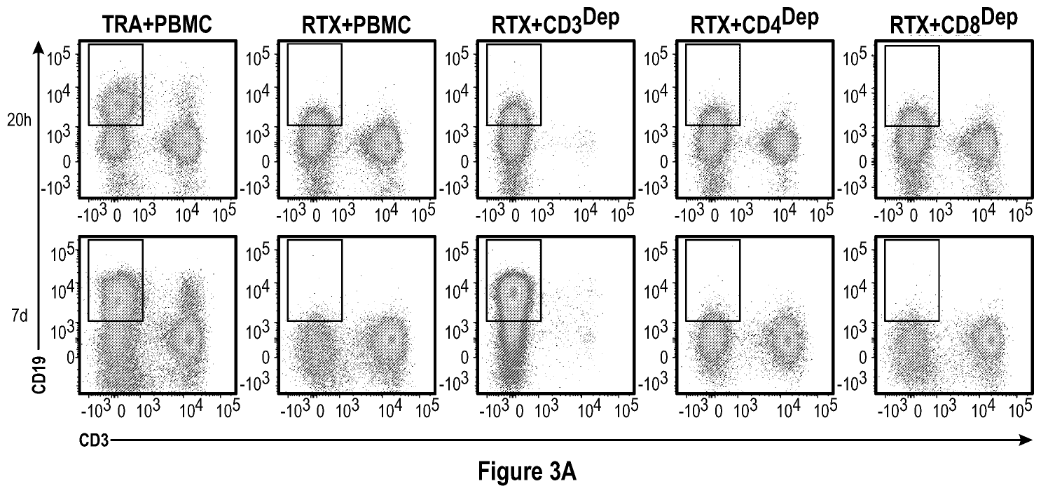

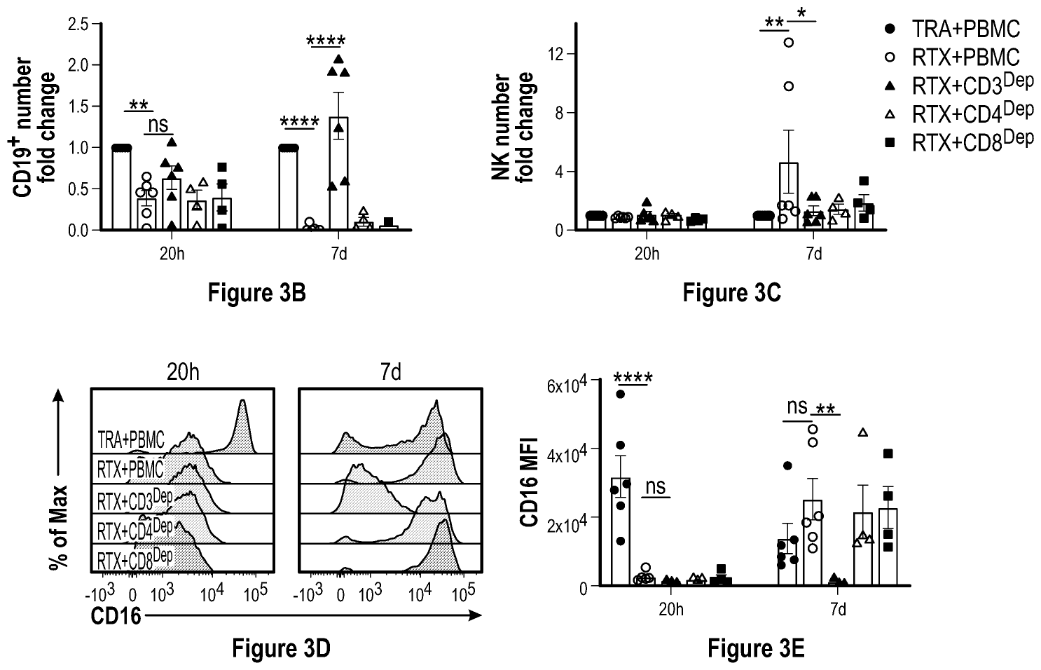

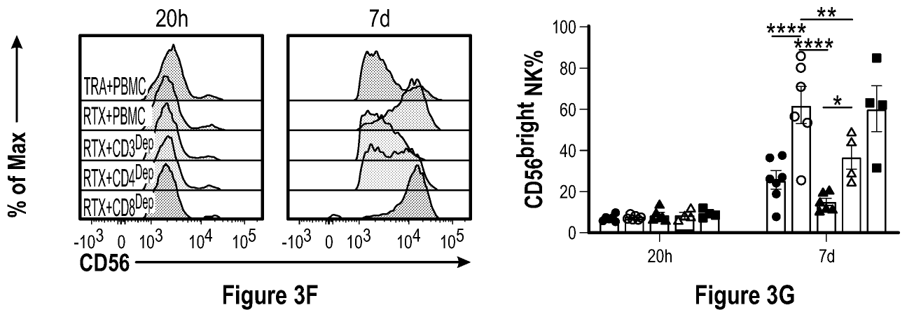

To identify the cellular component in PBMC supporting these changes, CD14⁺ monocytes, CD19⁺ B cells, CD3⁺, CD4⁺ or CD8⁺ T cells were depleted from PBMC and remaining cells cocultured with RTX and Raji cells. After 7 days, the depletion of CD3⁺ T cells inhibited RTX-mediated NK cell cytotoxicity, viability and CD16 re-expression (FIG. 3A-E). The depletion of CD3⁺ T cells or CD4⁺ T cells significantly suppressed the CD56^{dim} to CD56^{bright}NK transition after 7 days (FIG. 2F, G). Suppression of the CD56^{dim} to CD56^{bright} NK transition was most pronounced after CD3⁺ depletion, but was also seen with CD4⁺ depletion, suggesting CD4⁺ T cells are primarily responsible for supporting CD56^{dim} to CD56^{bright}NK transition but that CD8⁺ T cells can contribute to this process. The expression of activation markers on NK cells including CD25 and CD69 was not altered by the depletion of CD3⁺ T cells (FIGS. 10A, 10B). Depletion of benign B cells and monocytes had minimal impact on RTX-mediated NK cell cytotoxicity, viability, CD56^{dim} to CD56^{bright}NK transition or CD16 recovery after 7 days (FIGS. 11A-11G).

To further assess the role of T cells in RTX-mediated NK cell function and phenotype change, isolated NK cells were cocultured with RTX and Raji cells, and autologous CD3⁺, CD4⁺ or CD8⁺ T cells were added back before culturing for 7 days. RTX-mediated NK cell cytotoxicity was enhanced, and NK cell numbers were higher when CD3⁺ or CD4⁺ T cells were added back (FIGS. 12A-12C). CD56^{dim} to CD56^{bright} transition was not induced in isolated NK cells unless CD3⁺ T cells, CD4⁺ or CD8⁺ T cells were added back. CD3⁺ and CD4⁺ T cells triggered more CD56^{dim} to CD56^{bright} NK transition than did CD8⁺ T cells (FIGS. 12D, 12E). CD16 recovery was only seen after culture for 7 days with CD3⁺ or CD4⁺ T cells (FIGS. 12F, 12G). Taken together, these data demonstrate that T cells, largely CD4⁺ cells, are required to maintain RTX-mediated NK cell cytotoxicity, viability, CD56^{dim} to CD56^{bright} NK transition and CD16 recovery.

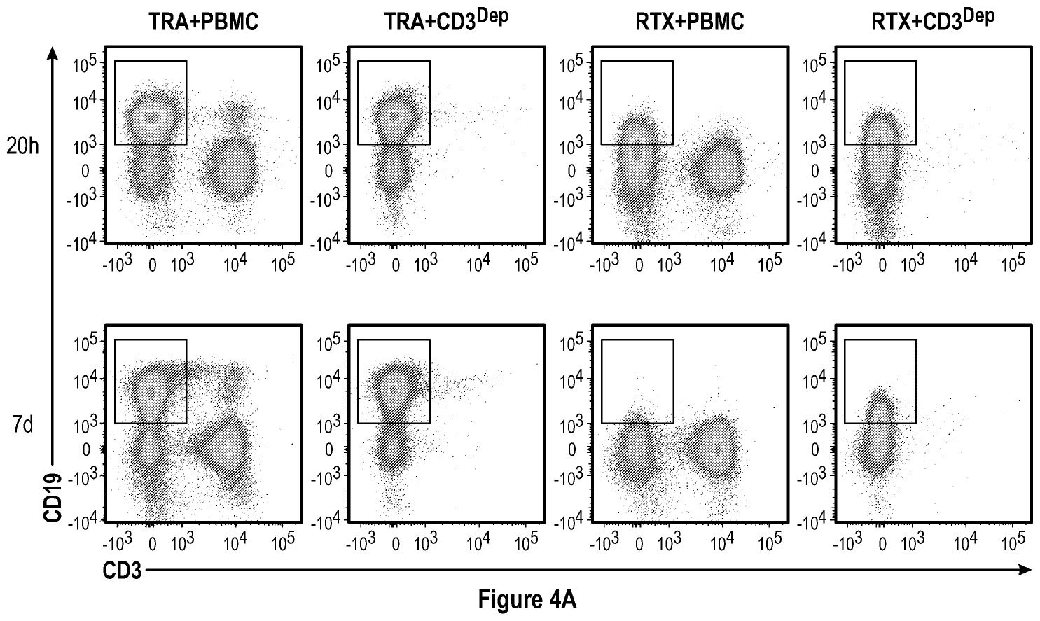

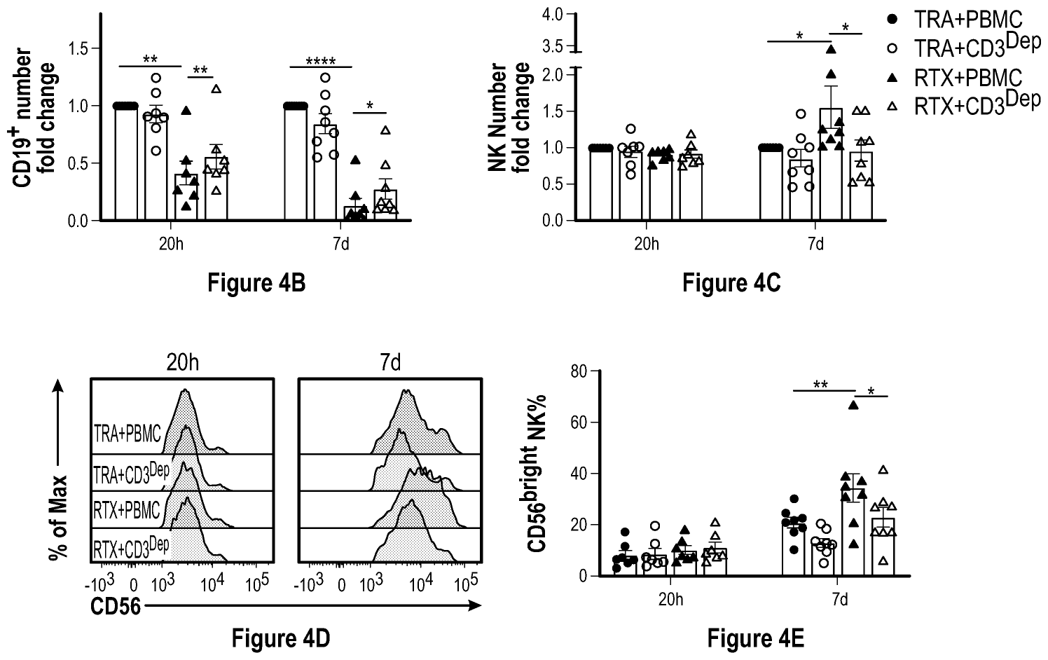

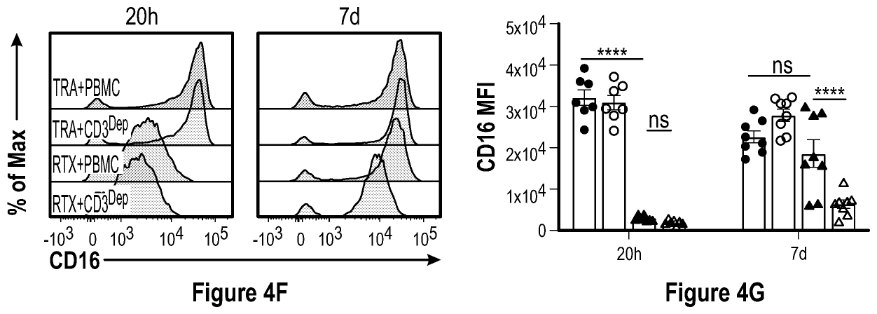

It is possible an allogeneic reaction between T cells and Raji cells contributed to changes in the NK cell responses. To assess this possibility, RTX was added to PBMCs enriched for autologous B cells that served as target cells for RTX and cultured for 7 days. Results in this fully autologous system were similar to those seen with Raji as target cells. RTX-mediated NK cell ADCC of autologous B cells, and NK viability was suppressed by the depletion of CD3⁺ T cells (FIG. 4A-C). CD56^{dim} to CD56^{bright} transition and CD16 re-expression were induced on NK cells in unfractionated PBMC but not after depletion of CD3⁺ T cells (FIG. 4D-G). The depletion of T cells did not impact CD25 or CD69 expression on NK cells (FIGS. 10C, 10D).

### IL2 in the Immunological Synapse Contributes to the Impact of T Cells on NK Cell Function

A Transwell system was used to investigate whether the impact of T cells on RTX-activated NK cells was contact dependent. When CD3⁺ T cells were physically separated from NK cells by the Transwell insert, the elimination of Raji cells, NK cell viability, CD56^{dim} to CD56^{bright} transition and CD16 recovery on NK cells was significantly reduced after 7 days (FIG. 5A-D). This suggests close contact between NK cells and T cells is needed to maintain the RTX-mediated NK cell response. Importantly, this does not exclude the possibility that soluble factors secreted by T cells impact on RTX-mediated NK cell response via the immunological synapse or in the close proximity of the cells.

T cells are known to interact with NK cells via a variety of ligand-receptor pairs including IL2-TL2R, IFNg-IFNgR, CD54-LFA1 and FGFR1-CD56. To investigate the mechanism of action by which T cells impact RTX-mediated NK cell responses, neutralization mAbs were used to block the potential NK-T cell interaction mediated by IL2, IFNg, CD54 and FGFR1. Anti-IL2 significantly inhibited RTX-mediated NK cell ADCC, viability, CD56^{dim} to CD56^{bright} transition and CD16-reexpression (FIG. 5E-H), suggesting IL2 was a critical mediator of T cells and RTX-activated NK cells. In addition, recombinant IL2 was added to the coculture of Raji cells and T-cell depleted PBMCs. IL2 supplementation was sufficient to maintain RTX-mediated NK cell ADCC, viability, CD56^{dim} to CD56^{bright} transition and CD16 re-expression (FIG. 5I-L). Therefore, T cells impact RTX-mediated NK cell response at least partially via IL2. The need for cell-cell contact suggests this interaction may take place in the immunological synapse.

The importance of CD16 in RTX efficacy has been demonstrated both in vitro and in patients. T-cell dependent changes in NK cell phenotype and function, including RTX-mediated NK cell cytotoxicity, viability and CD56^{dim} to CD56^{bright} transition, were suppressed by CD16 blockade using polyclonal anti-CD16 Ab (FIGS. 13A-13D). This confirms CD16 signaling is essential for RTX-mediated changes in NK cell function and phenotype.

### T Cells Maintain CTX-Mediated NK Cell Responses

To assess whether the observations outlined above are limited to anti-CD20 mAb or B cells as target cells, similar studies were done evaluating changes of NK cells in response to CTX, an anti-EGFR mAb, and EGFR expressing tumor cells. CTX induced NK cell CD56^{dim} to CD56^{bright} transition, maintained NK cell numbers and promoted CD16 recovery on NK cells (FIGS. 14A-14E) in a manner consistent with that seen with RTX. CTX-mediated effects on NK cells were dependent on the presence of CD3⁺ T cells and IL2 just as was seen with RTX (FIG. 14F-14H). This indicates that T cells may be critical in maintaining the long-term NK cell response to a variety of mAb via IL2.

### T Cell Activation Enhances RTX-Mediated NK Cell Function

Studies were done to determine whether activation of T cells enhances their ability to support NK cells. T cell activation by anti-CD3/CD28 beads using intact PBMCs did not alter RTX-mediated NK cell phenotype or function (FIG. 6A-D). T cells account for approximately 50% of PBMC. These data suggest resting T cells in large concentrations are able to provide T cell help to NK cells and this help is not enhanced by T cell activation. To more accurately mimic the lower T cell numbers in most tumor microenvironments, T cells were depleted from PBMC. Autologous resting T cells and T cells activated by anti-CD3/CD28 beads were then added back in various concentrations. RTX-mediated NK cell cytotoxicity, NK persistence, CD56^{dim} to CD56^{bright} transition, and CD16 recovery were all T cell dose-dependent (FIG. 6E-H). Activation of T cells enhanced NK cell responses particularly at lower doses of T cells. In some cases, NK cells changes were supported by activated T cell concentrations below 1%. This demonstrated activation of small numbers of T cells can enhance RTX-mediated NK cell responses. Similar results were found following addition of a bispecific anti-HLA-DR/anti-CD3 monoclonal antibody developed many years ago in our laboratory¹⁶ designated IDT3D (FIGS. 15A-15D).

### The Effects of T Cells on RTX-Activated NK Cell Transcriptomics

Bulk NK cell mRNA sequencing was used to evaluate the effects of T cells on the RTX-mediated NK cell response at the transcriptional level. PBMCs (unfractionated and after T cell depletion) were cultured for 7 days with RTX and Raji cells. NK cells were then isolated from three experimental conditions: (1) 0 h, resting PBMC (NK_naive), (2) Intact PBMCs (NK_PBMC), (3) T cell-depleted PBMC (NK_TCell_Dep). Transcriptomics of NK cells from the three groups were well distinguished from each other by principal component analysis (FIG. 7A), indicating they were transcriptionally different. A prime focus for analysis was on how T cells impact on RTX-mediated NK cell transcriptomics (FIG. 7B, C). Genes with enhanced expression in the NK_PBMC sample compared to the NK_TCell_Dep sample mapped to proliferation-associated biological processes including cell communication, cell division, cell cycle and chromosome segregation (FIG. 7D). The depletion of T cells did not influence the Fcg receptor signaling pathway or the NK cell cytotoxic pathway suggesting T cells have minimal impact on RTX-mediated NK cell activation (FIG. 7E). This analysis further supports the experimental findings that T cells impact on RTX-mediated NK cell response mainly by enhancing NK cell viability and proliferation, not by enhancing the cytotoxic potential of the NK cells.

### Discussion

The demonstration over 20 years ago that a mAb that targets a malignant cell, RTX, has significant and prolonged clinical anti-cancer activity has shifted the paradigm for treatment of B cell malignancies and other cancers. mAbs are now a standard component of treatment regimens for multiple cancers. Despite the remarkable success of anti-CD20 and other mAbs that directly target cancer, resistance and relapse remain common. RTX and other mAbs have remarkably long half-lives as drugs, with therapeutic levels being distributed in both the intravascular and extravascular compartments for weeks or even months during a course of therapy. The possible vaccinal effect of RTX suggests RTX impacts on the interaction between T cells and NK cells. This led us to explore the role of T cells in the long-term NK cell response to RTX. The overall goal of these studies was to enhance our understanding of the crosstalk between innate and adaptive immunity in the context of anti-tumor antibodies that could lead to the development of improved mAb-based therapeutics.

Proliferation of NK cells in response to anti-CD20 mAb has been previously reported in CLL samples when PBMCs containing NK cells, target cells and mAb are present together in the peripheral blood. The results presented here that mAb can induce proliferation of NK cells are consistent with these findings. The classic model for NK cells is that immature NK cells are CD56^{bright} and CD161^{low}, and that these cells mature into NK cells that are CD56^{dim} and CD16^{high} Growing evidence, including the data presented here, suggests a greater degree of complexity in the link between NK cell phenotype and biology. Previous studies reported that CD56^{dim} NK cells increase expression of CD56 in response to various stimuli including the Bacillus Calmette-Guerin vaccine, engineered antigen presentation cells, and cytokines. We found long-term activation of NK cells by mAb-coated target cells also induced increased expression of CD56. Furthermore, RTX-activated CD56^{dim} NK cells not only upregulate CD56, they also re-express CD16, express other maturation markers, and effectively mediate ADCC.

Surprisingly, the NK cell ability to mediate high levels of ADCC, viability, CD56^{dim} to CD56^{bright} transition, and re-expression of CD16 induced by RTX were seen with unfractionated PBMC but not with isolated NK cells, suggesting the interaction with other cell types plays a central role. Both cellular depletion and cell addback demonstrated that T cells, mainly CD4⁺, were responsible for this effect. The role of T cells in anti-CD20 efficacy has been evaluated in mouse models with a focus on eventual development of an anti-lymphoma T cell response. To our knowledge, our demonstration that T cells, particularly CD4⁺ cells, contribute to maintaining the viability of NK cells, thereby enhancing their ability to mediate ADCC, is a novel finding. Studies at the transcriptional level demonstrated T cells impact on differential expression of NK cell genes involved in NK cell viability and proliferation, with less of an impact on genes involved in NK cell activation or ability to mediate cytotoxicity. This finding is consistent with cellular and phenotypic analyses demonstrating that T cells impact on RTX-induced viability and proliferation of NK cells but not expression of NK activation markers at the protein level. Together, these data suggest T cells support persistence of activated NK cells activated by mAb as opposed to enhancing the ability of those NK cells to mediate ADCC. Results from Transwell assays suggest that close contact with NK cells is required for T cells to provide help and preserve the proliferation and viability of RTX-activated NK cells. Blocking the known ligand-receptor pairs of NK-T cell interaction demonstrated IL2 plays an important role in the ability of T cells to support RTX-mediated NK cell function and phenotypical changes. This finding is consistent with studies of vaccination and infection where NK cell activation has been found to be dependent on T cell-derived IL2. Use of IL2 to activate NK cells and enhance the efficacy of RTX and other mAb therapy is not a new concept. For example, IL2 was combined with RTX to in patients with relapsed and RTX-refractory follicular lymphoma in a clinical trial but failed to show synergistic efficacy. IL2 can activate and expand regulatory T (Treg) cells, which constitutively express the high-affinity IL2 receptor and suppress NK function. Perhaps more importantly, the kinetics and toxicity of exogenously administered IL2 would be expected to be very different than that of IL2 produced in the tumor microenvironment by resident T cells. It is therefore possible inducing production of IL2 by T cells in the tumor microenvironment will have a therapeutically important impact on NK cell mediated ADCC even though exogenously administered IL2 did not.

Changes in RTX-mediated NK cells were dependent on T cell dose. Furthermore, activated T cells were more effective than resting T cells at providing T cell help to NK cells. This suggests one potential mechanism of resistance to mAb therapy is lack of an adequate number of intratumoral T cells to provide T cell help that maintains the ability of NK cells to mediate ADCC. The finding that intratumor T cell infiltration correlates with better prognosis in lymphoma patients who have received RTX-containing therapy is consistent, although obviously does not prove, this hypothesis. A number of approaches could be used to activate intratumoral T cells with the goal of enhancing T cell help provided to NK cells. One such approach would be to combine standard mAb therapy with bispecific antibodies that can activate intratumoral T cells. Such combinations have been proposed before, but not based on the mechanism described here. Understanding this mechanism could impact on the design of such a regimen since the role of the bispecific antibody would not be to induce T cell mediated killing of the target cell directly. Instead, it would be to provide enough T cell help to support the NK cells that mediate ADCC. Intermittent dosing of the bispecific antibody, perhaps on a schedule similar to that for RTX or other mAb, might be adequate to achieve this goal. This would help overcome some of the major challenges associated with current approaches to retargeting T cells with bispecific antibodies that have been known for decades such as the need for continuous infusion and toxicity associated with cytokine storm.

In conclusion, contact of T cells, particularly activated CD4⁺ cells, with NK cells promotes mAb-induced phenotypic change, and maintains their viability and ability to mediate ADCC after long term in vitro culture. These findings suggest maintaining intratumoral T cell activation could enhance NK cell viability and ADCC thereby improving the efficacy of anti-CD20, and other mAb therapies where NK-mediated ADCC is a primary mechanism of action.

### Example 2

### T Cells are Required to Maintain Anti-CD20-Mediated NK Cell Responses In Vitro

Purpose: Anti-CD20 monoclonal antibodies, beginning with rituximab (RTX), are a mainstay of therapy for B cell malignancies. Despite their remarkable clinical efficacy, many patients fail to respond or develop resistance to anti-CD20 containing therapy. A better understanding of the mechanisms by which anti-CD20 antibodies mediate their anti-tumor effects is critical if we are to build further on what is already a remarkable success story.

Experimental procedures: Various subsets of healthy donor peripheral blood mononuclear cells (PBMCs) were cocultured in vitro with Raji cells and RTX or trastuzumab (TRA) as a control antibody for up to 7 days. The number of remaining Raji cells, NK cells and phenotype of NK cells was determined.

Results: When unfractionated PBMCs were cultured with Raji cells for 1 week, RTX depleted Raji cells and enhanced NK cell numbers compared to TRA. In such cultures, RTX also induced changes in NK cell phenotype including a shift from CD56^{dim} to CD56^{bright} and increased expression of CD16, CD57 and KIR. These changes were dependent on T cells. Depletion of T cells, largely CD4, suppressed elimination of Raji cells, NK cell viability and the change in NK cell phenotype. Cell to cell contact was required, as the ability of T cells to support NK cell cytotoxicity, viability and phenotypic change was lost when T cells were separated from other cells in a transwell assay. T cell activation with anti-CD3/CD28 beads enhanced the ability of T cells to support RTX-mediated NK cell responses, with concentrations of activated T cells as low as 1% being adequate to provide T cell help to NK cells. Similar results were seen when obinutuzumab was used instead of RTX and when autologous B cells were used as target cells instead of Raji cells demonstrating the observed T cell effect was not secondary to an allogenic response by T cells to Raji cells.

Conclusion: In longer term in vitro culture, anti-CD20 induces lysis of target B cells, preserves NK cell viability, and induces CD56^{dim} to CD56^{bright} transition and CD16 recovery in a T cell dependent manner. This T cell help requires cell-to-cell contact with NK cells and is enhanced following T cell activation. Ongoing studies are exploring the precise mechanisms by which T cells support NK cells, and whether similar changes are observed in patients. These findings could lead to evaluation of a combination of anti-CD20 therapy and T cell activation as a strategy for overcoming resistance to ant-CD20 therapy.

### Example 3

### Bispecific Antibody-Activated T Cells Enhance NK Cell-Mediated Antibody-Dependent Cellular Cytotoxicity

Resistance to anti-cancer monoclonal antibody (mAb) therapy remains a clinical challenge. Previous work has shown that T cell help in the form of interleukin-2 maintains long-term NK cell viability and NK cell mediated antibody-dependent cellular cytotoxicity (ADCC). Lack of such T cell help may be a potential mechanism for resistance to mAb therapy. Here, it was evaluated whether concomitant treatment with anti-CD3×anti-cancer bispecific antibodies (bsAbs) can overcome this resistance by enhancing T cell help, and thereby maintaining long-term NK cell-mediated ADCC. Normal donor peripheral blood mononuclear cells were depleted of T cells, replenished with defined numbers of autologous T cells (from 0.75 to 50%) and co-cultured with mono-/bispecific antibody-treated target tumor cells for up to 7 days. At low T cell concentrations, bsAb-activated T cells (mainly CD4⁺ T cells) were more effective than resting T cells at maintaining NK cell viability and ADCC. Brief (4 h to 2 day) bsAb exposure was sufficient to enhance long-term ADCC by NK cells. These findings raise the hypothesis that local T cell activation mediated by systemic treatment with anti-CD3×anti-cancer bsAb may enhance the anti-tumor efficacy of monospecific mAbs that mediate their primary therapeutic effect via NK-mediated ADCC.

Anti-cancer monoclonal antibodies (mAbs), including rituximab (anti-CD20) and cetuximab (anti-EGFR), are a standard component of cancer therapy. A major mechanism of action of anti-cancer mAbs is NK cell-mediated antibody-dependent cellular cytotoxicity (ADCC). Resistance to mAb therapy remains a clinical challenge. Our previous work suggests that T cell help, mediated largely by interleukin-2 (IL-2) locally produced by CD4⁺ T cells, maintains long-term NK cell-mediated ADCC and NK number. Thus, lack of adequate T cell help may explain some cases of resistance to mAb therapy.

IL-2 is well known to enhance NK cell activation and ADCC. However, systemic IL-2 administration results in significant toxicity and non-selectively expands regulatory T cells, thereby lessening enthusiasm for such combinations. Anti-CD3×anti-cancer bispecific antibodies (bsAbs) redirect T cell cytotoxicity towards tumor cells. bsAb-activated T cells also produce proinflammatory cytokines, including IL-2. Here, the hypothesis was explored that bsAb can induce the local production of IL-2 by T cells and maintain NK cell-mediated ADCC.

T cells were depleted from peripheral blood mononuclear cells (PBMCs) and autologous T cells were added back in known concentrations along with target Raji cells, rituximab (RTX) and blinatumomab (anti-CD19×anti-CD3) and cultured for 1 week. Blinatumomab at either 1 or 10 ng/mL enhanced elimination of Raji cells by RTX-activated NK cells (FIG. 16A) and increased the number of viable NK cells (FIG. 16B), particularly when lower numbers of T cells were present. By contrast, RTX or blinatumomab alone had minimal impact on NK cells or ADCC when small numbers of T cells were present. The addition of T cells in the trastuzumab control group had little effect on CD19⁺ cell numbers indicating nutrient depletion was not responsible for limiting Raji growth. These results demonstrate that small numbers of T cells activated by blinatumomab enhance RTX-mediated ADCC and NK cell number. The number of viable NK cells was lower in response to RTX plus blinatumomab compared to RTX alone at high T cell concentrations, likely due to the early elimination of target cells and the loss of the RTX-mediated activating signal to NK cells. Concentrations of blinatumomab below 1 ng/ml had limited impact on RTX-mediated NK cell responses (FIGS. 16C, 16D). Similar results were observed with Daudi cells serving as target cells. Both CD4⁺ and CD8⁺ T cells were able to produce IL-2 in response to blinatumomab. Blinatumomab-activated CD4⁺ and CD8⁺ T cells enhanced NK cell ADCC and number, with CD4⁺ T cells being more effective at low T cell concentrations.

One major challenge associated with the clinical use of blinatumomab is the need for a continuous 28-day infusion. This led to evaluation of whether short-term blinatumomab exposure can provide enough T help to support RTX-mediated NK cell ADCC. Blinatumomab was added to the culture for various periods of time, then washed out while RTX was maintained for the full 7 days (FIG. 17A). Short-term (4-h or 2-day) blinatumomab exposure enhanced NK cell ADCC and number (FIGS. 17B, 17C). A similar IL-2 washout experiment (FIG. 17D) was performed to explore the role of IL-2 in this process. These results (FIGS. 17E, 17F) are consistent with the prior observation that TL-2 production by T cells is central to providing help for NK-mediated ADCC. Additionally, EGFRBi (anti-EGFR×anti-CD3) enhanced cetuximab-mediated NK cell ADCC, suggesting bsAb-induced T cell help can enhance ADCC mediated by mAb against other targets.

Collectively, these studies describe a novel mechanism of interaction between mAbs and bsAbs that has the potential to enhance the therapeutic effectiveness of both agents. Anti-CD3× anti-cancer bsAb induces the local production of cytokines, including IL-2 by T cells which, in turn, enhances NK cell viability and ability to mediate ADCC.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

Further, all numbers expressing quantities of ingredients, reaction conditions, % purity, polypeptide and polynucleotide lengths, and so forth, used in the specification and claims, are modified by the term “about,” unless otherwise indicated. Accordingly, the numerical parameters set forth in the specification and claims are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention. Further, all publications mentioned herein are incorporated by reference in their entireties.

1. A method for treating a hyperproliferative disorder in a mammal in need thereof comprising, administering to the mammal a combination of (a) an anti-cancer antibody and (b) a T cell activating agent for the prophylactic or therapeutic treatment of the hyperproliferative disorder.

2. The method of claim 1, wherein the anti-cancer antibody mediates antibody-dependent cellular cytotoxicity (ADCC).

3. The method of claim 1, wherein the ADCC is mediated by natural killer (NK) cells.

4. The method of claim 1, wherein the anti-cancer antibody is a monospecific antibody.

5. The method of claim 4, wherein the monospecific antibody is Margetuximab, Naxitamab, Tafasitamab, Isatuximab, Mogamuizumab, Olaratumab, Daratumumab, Elotuzumab, Necitumumab, Dinutuximab, Ramucirumab, Obinutuzumab, Pertuzumab, Ofatumumab, Panitumumab, Cetuximab, Alemtuzumab, Trastuzumab, Rituximab, or Edrecolomab.

6. The method of claim 1, wherein the T cell activating agent activates CD4⁺ T cells.

7. The method of claim 1, wherein the T cell activating agent is a bispecific antibody, a chimeric antigen receptor T (CAR-T) cells, and/or a vaccine that induces a T cell response.

8. The method of claim 7, wherein the T cell activating agent is a bispecific antibody.

9. The method of claim 7, wherein the T cell activating agent is a bispecific anti-CD3×anti-tumor antibody.

10. The method of claim 9, wherein the bispecific anti-CD3×anti-tumor antibody is Blinatumomab or Catumaxomab.

11-13. (canceled)

14. The method of claim 1, wherein the hyperproliferative disorder is cancer.

15. The method of claim 14, wherein the cancer is selected from the group consisting of B cell lymphoma, T cell lymphoma, myeloma, non-small cell lung cancer, small cell lung cancer, breast cancer, head and neck cancer, neuroblastoma, soft tissue sarcoma, gastric cancer, colorectal cancer, chronic lymphocytic leukemia, acute lymphoblastic leukemia, chronic myeloid leukemia, acute myeloid leukemia, pancreatic cancer, and prostate cancer.

16. A method for maintaining long-term natural killer (NK) cell antibody-dependent cellular cytotoxicity (ADCC) in the treatment of a hyperproliferative disorder in a mammal in need thereof comprising, administering to the mammal a combination of (a) an anti-cancer antibody, and (b) a T cell activating agent for the prophylactic or therapeutic treatment of the hyperproliferative disorder.

17. The method of claim 1, wherein the anti-cancer antibody and the T cell activating agent are administered separately, simultaneously or sequentially.

18. The method of claim 17, wherein the anti-cancer antibody and the T cell activating agent are administered simultaneously.

19. The method of claim 1, wherein the administration is repeated weekly.

20. The method of claim 1, wherein the administration is repeated monthly.

21. (canceled)

22. The method of claim 1, wherein the T cell activating agent is administered intravenously or subcutaneously.

23. The method of claim 1, wherein the T cell activating agent is administered at a dose of between about 2 and 50 micrograms.

24-25. (canceled)

26. A kit comprising a T cell activating agent, a container, and a package insert or label indicating the administration of the T cell activating agent with an anti-cancer antibody for treating a hyperproliferative disorder.

27. (canceled)
